(19) United States
(12) Patent Application Publication
(10) Pub. No.: US 2026/0102438 A1
(43) Pub. Date: Apr. 16, 2026

(54) Bacterial Extracellular Vesicles as Novel Therapeutic Agents for Disease Treatment

(71) Applicant: University of Kansas, Lawrence, KS (US)

(72) Inventor: Jun Zhang, Overland Park, KS (US)

(21) Appl. No.: 19/355,672

(22) Filed: Oct. 10, 2025

Related U.S. Application Data
(60) Provisional application No. 63/705,859, filed on Oct. 10, 2024.

(51) Int. Cl. A61K 35/74; A61K 39/395

(52) U.S. Cl. CPC A61K 35/74; A61K 39/39558

(57) ABSTRACT

A composition may include an anti-cancer therapeutic agent and a bacteria-derived extracellular vesicle (BEV). The anti-cancer therapeutic agent may include an alkylating agent, an antimetabolite, a plant alkaloid, an antitumor antibiotic, a topoisomerase inhibitor, a hormonal agent, a targeted therapeutic, an immunotherapy agent, a monoclonal antibody, a proteasome inhibitor, an epigenetic modifier, hydroxyurea, or L-asparaginase. The BEV may be derived from commensal bacteria including gut microbiota commensal bacteria, skin microbiota commensal bacteria, oral microbiota commensal bacteria, respiratory tract microbiota commensal bacteria, urogenital microbiota commensal bacteria, or nasal microbiota commensal bacteria. The BEV may be derived from one or more Bifidobacterium species. The composition may include a pharmaceutically acceptable carrier, additive, excipient, or other component of a pharmaceutical composition. The BEV may include lipoteichoic acid (LTA).

### CROSS REFERENCE TO RELATED APPLICATIONS

This patent application claims priority to U.S. Provisional Application No. 63/705,859 filed Oct. 10, 2024, which is incorporated herein by reference in its entirety.

### TECHNICAL FIELD

The present disclosure relates to bacterial extracellular vesicles (BEVs) as novel therapeutic agents for disease treatment.

### BACKGROUND

Immunotherapy approaches, including administration of anti-programmed death-1 (anti-PD-1) and anti-programmed death ligand 1 (anti-PD-L1) immune checkpoint inhibitors, may be employed as standard treatment for diseases, such as non-small cell lung cancer (NSCLC). However, a considerable fraction of patients may not experience effective clinical benefit from such immunotherapy approaches. Studies have identified potential strategies to enhance antitumor immune responses by modulating microbiota. Despite these advances, the mechanisms by which microbiota influence disease microenvironments and alter immunotherapy outcomes remain insufficiently understood.

### SUMMARY

In some embodiments, a composition may include an anti-cancer therapeutic agent and a bacteria-derived extracellular vesicle (BEV). In these and other embodiments, the anti-cancer therapeutic agent may include one or more of an alkylating agent, an antimetabolite, a plant alkaloid, an antitumor antibiotic, a topoisomerase inhibitor, a hormonal agent, a targeted therapeutic, an immunotherapy agent, a monoclonal antibody, a proteasome inhibitor, an epigenetic modifier, hydroxyurea, or L-asparaginase. Additionally or alternatively, the BEV may be derived from commensal bacteria. In some embodiments, the BEV may be derived from one or more of gut microbiota commensal bacteria, skin microbiota commensal bacteria, oral microbiota commensal bacteria, respiratory tract microbiota commensal bacteria, urogenital microbiota commensal bacteria, or nasal microbiota commensal bacteria. In these and other embodiments, the BEV may be derived from one or more Bifidobacterium species. In some embodiments, the composition may further comprise a pharmaceutically acceptable carrier, additive, excipient, or other component of a pharmaceutical composition. In some embodiments, the BEV may include lipoteichoic acid (LTA).

In some embodiments, a method of treating a disease may include administering a BEV to a subject having a disease in an amount sufficient to treat the disease. In these and other embodiments, the disease may be cancer. In some embodiments, the BEV may be administered with an anti-cancer therapeutic agent. In some embodiments, the BEV may be derived from commensal bacteria, such as gut microbiota commensal bacteria, skin microbiota commensal bacteria, oral microbiota commensal bacteria, respiratory tract microbiota commensal bacteria, urogenital microbiota commensal bacteria, and/or nasal microbiota commensal bacteria. In these and other embodiments, the BEV may be derived from a Bifidobacterium species. Additionally or alternatively, the BEV may be configured to regulate an immune checkpoint. In some embodiments, the BEV may be configured to inhibit upregulation or decrease regulation of programmed death-ligand 1 (PD-L1). In these and other embodiments, the BEV may be administered with an anti-PD-L1 immune checkpoint inhibitor. In some embodiments, the BEV and the anti-PD-L1 immune checkpoint inhibitor may cause modulated cytokine levels and/or increased tumor infiltrating lymphocytes. Additionally or alternatively, the BEV may be configured to cause one or more of upregulation of major histocompatibility complex (MHC) peptide, increased interferon response, increased toll-like receptor 4 (TLR4) signaling, and/or increased inhibition of angiogenesis.

In some embodiments, a method of increasing antitumor immune response in a subject with a disease may include administering an effective amount of BEVs and anti-PD-L1 immune checkpoint inhibitor. In some embodiments, the disease may be cancer. In some embodiments, the BEV may be derived from commensal bacteria. In these and other embodiments, the BEV may be derived from gut microbiota commensal bacteria, skin microbiota commensal bacteria, oral microbiota commensal bacteria, respiratory tract microbiota commensal bacteria, urogenital microbiota commensal bacteria, or nasal microbiota commensal bacteria. In some embodiments, the BEV may be derived from a Bifidobacterium species. Additionally or alternatively, administering the effective amount of the BEV and the anti-PD-L1 immune checkpoint inhibitor may cause modulated cytokine levels and/or increased tumor infiltrating lymphocytes.

In some embodiments, a kit for treating lung cancer may include commensal Bifidobacterium-derived extracellular vesicles (Bif.BEVs) and one or more of an anti-programmed death 1 (anti-PD-1) immune checkpoint inhibitor or an anti-PD-L1 immune checkpoint inhibitor. In some embodiments, the Bif.BEVs may include LTA. In these and other embodiments, the Bif.BEVs may be derived from gut microbiota commensal bacteria, skin microbiota commensal bacteria, oral microbiota commensal bacteria, respiratory tract microbiota commensal bacteria, urogenital microbiota commensal bacteria, and/or nasal microbiota commensal bacteria. In some embodiments, the anti-PD-1 immune checkpoint inhibitor may include nivolumab, pembrolizumab, cemiplimab, tislelizumab, dostarlimab, retifanlimab, and/or toripalimab. Additionally or alternatively, the anti-PD-L1 immune check point inhibitor may include one or more of atezolizumab, avelumab, durvalumab, and cosibelimab.

The foregoing summary is illustrative only and is not intended to be in any way limiting. In addition to the illustrative aspects, embodiments, and features described above, further aspects, embodiments, and features will become apparent by reference to the drawings and the following detailed description.

### BRIEF DESCRIPTION OF THE DRAWINGS

The patent or application file contains at least one drawing executed in color. Copies of this patent or patent application publication with color drawing(s) will be provided by the Office upon request and payment of the necessary fee.

The accompanying drawings, which form a part of the present disclosure, illustrate various embodiments and features as described in greater detail below.

FIGS. 1A-1D depict example characterization data of Bifidobacterium-derived extracellular vesicles (Bif.BEVs);

FIGS. 2A and 2B depict cellular uptake and programmed death ligand 1 (PD-L1) modulation by Bif.BEVs;

FIGS. 3A-3D depict the uptake of Bif.BEVs in various cell lines;

FIGS. 4A and 4B depict quantitative characterization data corresponding to Bif.BEVs;

FIG. 5 depicts data corresponding to a reduction of lipoteichoic acid (LTA) in dynamin-deficient cells after incubation of cells with Bif.BEVs;

FIGS. 6A-6C depict western blot analysis data corresponding to Bif.BEVs;

FIG. 7 depicts the uptake of Bif.BEVs by organoids;

FIG. 8 depicts Bif.BEV uptake by a spheroid developed from human lung adenocarcinoma tissue;

FIG. 9A depicts immunofluorescence imaging of organoids treated with Bif.BEVs;

FIG. 9B depicts human lung adenocarcinoma organoids treated with dynasore (DS) inhibitor;

FIG. 10 depicts example evaluation data of endocytosis inhibitors on Bif.BEV uptake in three-dimensional organoids;

FIG. 11 depicts in vivo images of a syngeneic mouse tumor model after oral administration of Bif.BEVs;

FIGS. 12A and 12B depict data indicating that Bif.BEVs enhance the therapeutic effect of anti-PD-1;

FIGS. 13A and 13B depict data indicating that Bif.BEVs enhance anti-PD-1 efficacy via modulation of cytokines;

FIGS. 14A and 14B depict data indicating that Bif.BEVs in combination with anti-PD-1 increase tumor infiltrating T lymphocytes;

FIGS. 15A and 15B depict data indicating that Bif.BEVs upregulate PD-L1 expression in tumor cells and modulate immune and oncogenic pathways; and

### DETAILED DESCRIPTION

Immunotherapy using immune checkpoint inhibitors (ICIs), such as those targeting programmed death 1 (PD-1), programmed death ligand 1 (PD-L1), and/or cytotoxic T-lymphocyte-associated protein 4 (CTLA-4), has emerged as a treatment of choice for the routine care of variety of diseases. For example, immunotherapy may be used to treat cancers including lung cancers, such as non-small cell lung cancer (NSCLC). Immunotherapy treatment has proven efficacious by prolonging patient survival. However, only a small proportion of patients directly derive benefit from immunotherapy. Additionally, immune related adverse events (irAEs) may limit the therapeutic value of such immunotherapy treatments. Finding modifiable modulating factors of immunotherapy treatments is therefore desirable and clinically relevant.

Microorganisms, such as gut microbiota, may modulate immunotherapy approaches. For example, commensal Bifidobacterium (Bif.B) in the gut may enhance the antitumor effects of anti-PD-L1 therapy. Additionally, Bif.B may mitigate intestinal immunopathology without affecting an antitumor response.

The tumor microenvironment (TME) or tumor immune microenvironment (TIME) may impact the response a patient has to one or more immunotherapy approaches. Since gut microbiota reside inside of the intestinal tract where cellular tight junctions and a mucin layer over the intestinal epithelium prevent gut microbiota from crossing the intestinal barrier, how gut microbiota remotely affect the TIME of cancer and responses to immunotherapy remains elusive. Bacterial metabolites (e.g., short chain fatty acids (SCFA)), migration of locally activated immune cells, and altered cytokines and/or chemokines have all been speculated to address the effect of gut microbiota on cancer immunotherapy.

All eukaryotic and prokaryotic cells constantly interact with their surroundings by means of cellular components, among which, the production, uptake, and secretion of extracellular vesicles (EVs) is one primordial feature. EVs may deliver numerous immune-stimulatory or modulatory signals from donor cells to recipient cells that may further regulate immune responses in mammalian host cells making them potential immunotherapeutic agents.

In some embodiments of the present disclosure, bacteria-derived extracellular vesicles (BEVs) may be derived from commensal bacteria that may include gut (i.e., intestinal) microbiota, such as Bacteroides species (e.g., Bacteroides fragilis, Bacteroides vulgatus), Firmicutes (e.g., Lactobacillus acidophilus, Clostridium butyricum, Faecalibacterium prausnitzii), Prevotella species (e.g., Prevotella copri), Akkermansia muciniphila, Eubacterium rectale, and/or Bifidobacterium species (e.g., Bifidobacterium longum, Bifidobacterium breve), among others. Additionally or alternatively, the BEV may be derived from skin microbiota commensal bacteria, oral microbiota commensal bacteria, respiratory tract microbiota commensal bacteria, urogenital microbiota commensal bacteria, and/or nasal microbiota commensal bacteria.

In some embodiments, BEVs may be used to connect gut microbiota remotely to the TIME of NSCLC. In these and other embodiments, BEVs derived from the Gram-positive bacteria Bif.B (Bif.BEVs) may remotely modulate the TIME of NSCLC and/or a response to anti-PD-1 immunotherapy.

In some embodiments, one or more systemic methods for studying Bif.BEVs in vitro, ex vivo, and/or in vivo may be used.

In some embodiments, Bif.BEVs may be used in conjunction with one or more anti-cancer therapeutic agents to treat various cancers including non-small cell lung cancer (NSCLC), melanoma, renal cell carcinoma, head and neck squamous cell carcinoma (HNSCC), urothelial carcinoma, and Hodgkin lymphoma, among others. In these and other embodiments, the anti-cancer therapeutic agent may be an alkylating agent (e.g., cyclophosphamide, ifosfamide, melphalan, chlorambucil, cisplatin, carboplatin, oxaliplatin, etc.); an antimetabolite (e.g., methotrexate, fluorouracil (5-FU), capecitabine, cytarabine, gemcitabine, mercaptopurine, fludarabine, etc.); a plant alkaloid (e.g., vincristine, vinblastine, vinorelbine, paclitaxel, docetaxel, topotecan, irinotecan, etc.); an antitumor antibiotic (e.g., doxorubicin, daunorubicin, bleomycin, mitomycin, dactinomycin, etc.); a topoisomerase inhibitor (e.g., etoposide, teniposide, irinotecan, topotecan, etc.); a hormonal agent (e.g., tamoxifen, letrozole, anastrozole, exemestane, leuprolide, goserelin, flutamide, etc.); a targeted therapeutic (e.g., imatinib, erlotinib, gefitinib, trastuzumab, bevacizumab, sorafenib, sunitinib, etc.); an immunotherapy agent (e.g., an anti-PD-1 antibody, an anti-PD-11 antibody, pembrolizumab, nivolumab, ipilimumab, atezolizumab, durvalumab, avelumab, rituximab, alemtuzumab, etc.); a monoclonal antibody (e.g., rituximab, trastuzumab, cetuximab, bevacizumab, daratumumab); a proteasome inhibitor (bortezomib, carfilzomib, etc.); an epigenetic modifier (e.g., vorinostat, romidepsin, azacitidine, decitabine, etc.); and/or any other anticancer agent (e.g., hydroxyurea, L-asparaginase, etc.).

In some embodiments, Bif.BEVs may upregulate major histocompatibility complex (MHC) peptide assembly and presentation, increase response to interferons (e.g., alpha and gamma), enhance T cell-mediated cytotoxicity, improve Toll-like receptor 4 (TLR4) signaling, and/or inhibit angiogenesis.

In the following detailed description, reference is made to the accompanying drawings, which form a part hereof. In the drawings, similar symbols typically identify similar components, unless context dictates otherwise. The illustrative embodiments described in the detailed description, drawings, and claims are not meant to be limiting. Other embodiments may be utilized, and other changes may be made, without departing from the spirit or scope of the subject matter presented herein. It will be readily understood that the aspects of the present disclosure, as generally described herein, and illustrated in the figures, can be arranged, substituted, combined, separated, and designed in a wide variety of different configurations, all of which are explicitly contemplated herein.

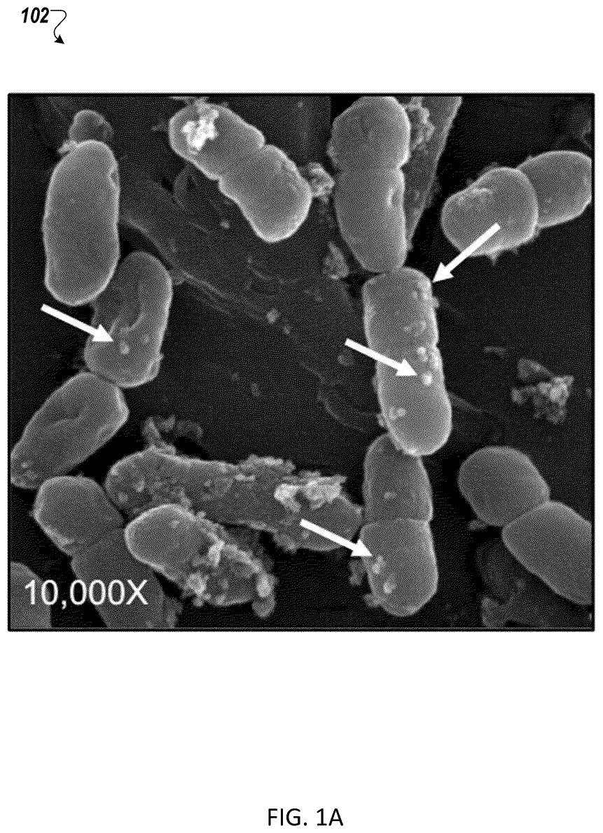

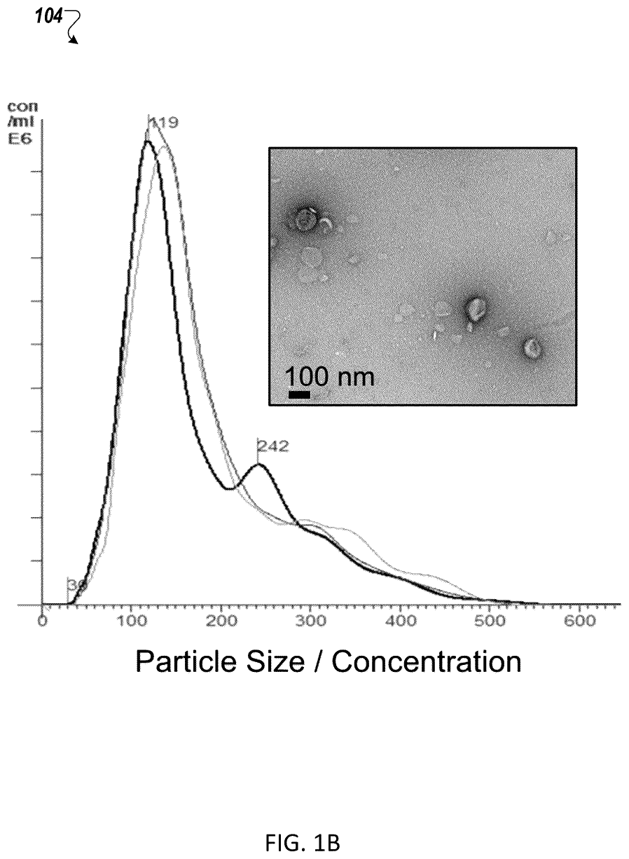

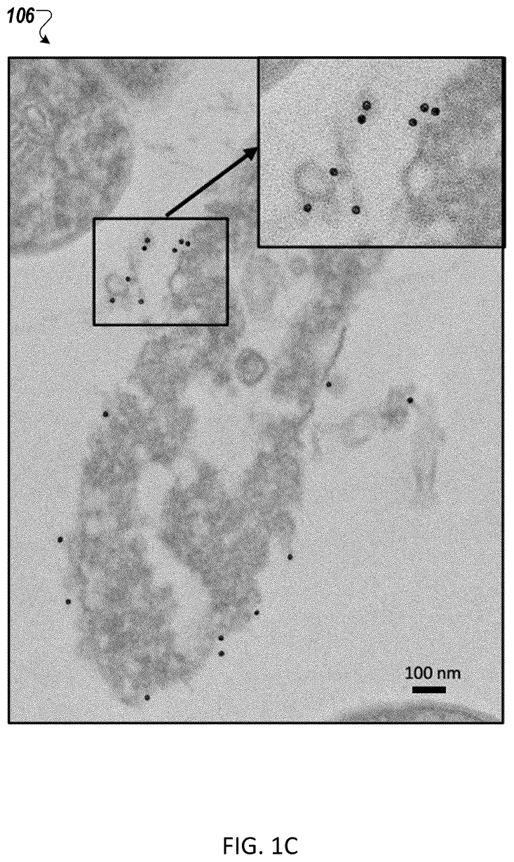

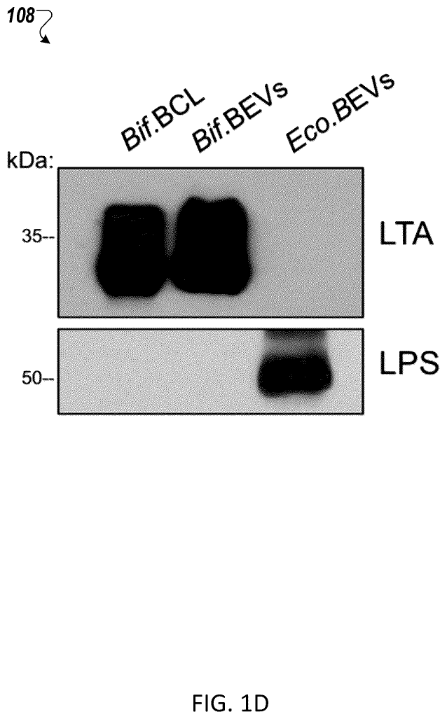

Turning to the figures, FIGS. 1A-1D depict example characterization data of Bif.BEVS. In some embodiments, Bif.BEVs may show characteristic EV features and carry lipoteichoic acid (LTA) of Bif.B origin. In these and other embodiments, Bif.BEVs may have a characteristic morphology, size range, and origin similar to mammalian-derived EVs. In some embodiments, the release of Bif.BEVs may be assessed and the released Bif.BEVs may be characterized. For example, as depicted in the scanning electron microscopy (SEM) image 102, SEM may be used to visualize the surface of Bif.B. SEM may show vesicle-like structures protruding/blebbing outside on the surface of Bif.B. In some embodiments, a significant number of Bif.B may secrete such vesicles.

To further characterize those secreted vesicles, Bif.B may be grown in anaerobic culture for 48 hours and then supernatant may be collected. In some embodiments, Bif.BEVs may be isolated by using an ultracentrifugation approach. The size of Bif.BEVs may be determined by nanoparticle tracking analysis (NTA) 104. In some embodiments, isolated Bif.BEVs may range from 30-500 nm with the majority of the Bif.BEVs falling in the 119 nm and 242 nm range. Isolated Bif.BEVs may be further subjected to transmission electron microscopy (TEM) to visualize Bif.BEVs and assess their morphology. In some embodiments, Bif.BEVs may show characteristic cup-shaped morphology. To assess that isolated BEVs originated from Bif.B, in some embodiments, immunogold labeling against surface associated LTA may be performed on the Bif.B and the sections of bacteria may be visualized by TEM to identify anti-LTA-gold particles on the bacterial surface. An immunogold TEM micrograph 106 may show the presence of LTA on the surface of bacteria. Additionally or alternatively, anti-LTA-immunogold labeling may be also observed on the vesicles (e.g., secreted Bif.BEVs) in the close vicinity of the Bif.B. To further validate the presence of LTA in Bif.BEVs, a western blot analysis 108 may be performed. LTA may be found present in lysates from both Gram-positive Bif.B cells and their BEVs (Bif.BEVs) but not from Gram-negative E. coli-derived EVs (Eco.BEVs). Anti-LPS western blot may be used to differentiate Gram-positive Bif.B and Bif.BEV lysates from Gram-negative Eco.BEV lysate.

In some embodiments, Bif.BEVs have characteristic morphology of BEVs and contain LTA as a component of their cargo. In some embodiments, LTA may be chosen as a marker for Bif.BEVs because LTA is a major component of the cell wall in Gram-positive bacteria, anchoring to the cell membrane and/or because LTA serves as a specific biomarker for BEVs derived from Gram-positive bacteria. LTA as a biomarker may be advantageous due to the availability of commercially available antibodies.

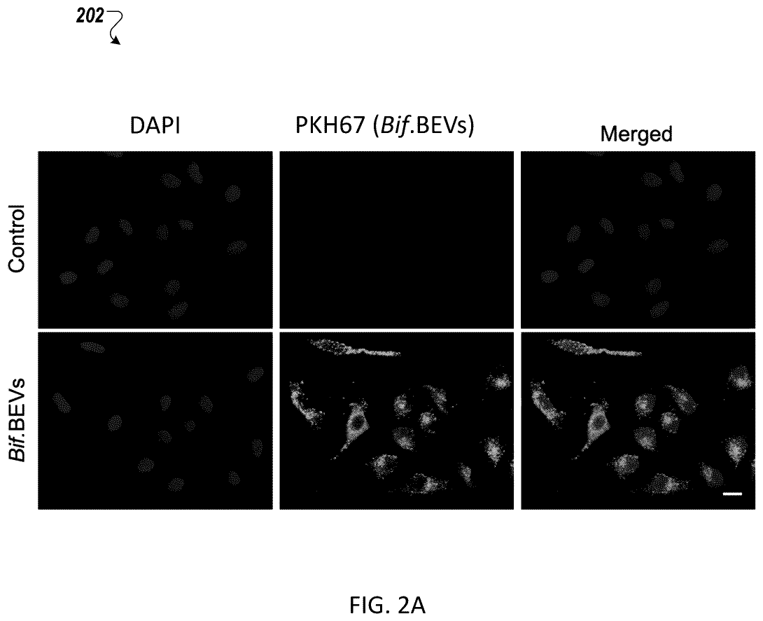

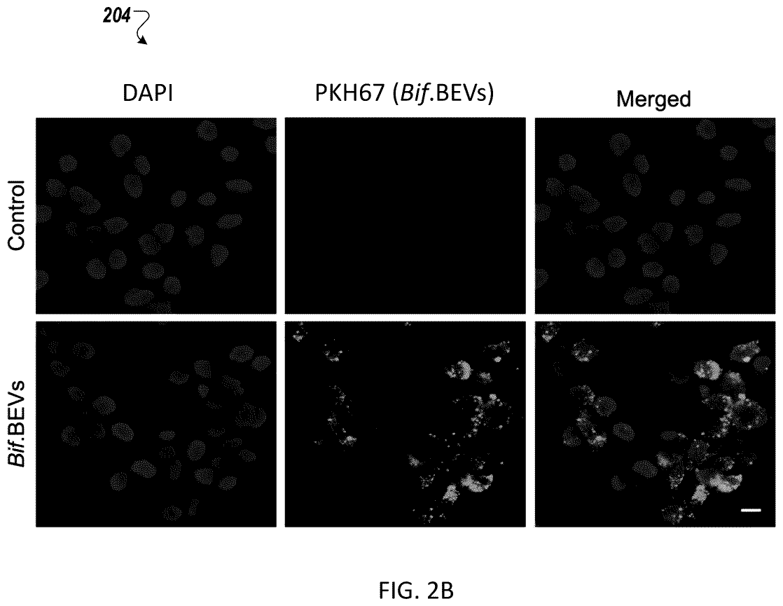

FIGS. 2A and 2B depict immunofluorescent detection data of Bif.BEV uptake by lung cancer cells. In some embodiments, the uptake of Bif.BEVs may utilize dynamin-dependent endocytosis and modulate the expression of immune checkpoints. Previous studies have lacked data to suggest Bif.BEVs may possess immunomodulatory capacity. The present disclosure includes investigation of such immunomodulatory capacity using a two-dimensional in vitro system. As shown in the first immunofluorescent detection data 202, human lung cancer A549 cells may be treated for 24 hours with isolated Bif.BEVs fluorescently labeled with PKH67, a membrane dye. Immunofluorescent imaging of cells demonstrated uptake of green PKH67-labeled Bif.BEVs in A549 cells. Similar uptake may be also observed using murine lung cancer LL/2 cells, as shown in the second immunofluorescent detection data 204.

To explore the possible mechanism of cellular uptake of Bif.BEVs, various inhibitors including amiloride (AM), cytochalasin D (CD), dynasore (DS) and nystatin (NS) may be used. AM inhibits the Na+/H+ exchanger that is crucial for macrocytosis; CD inhibits actin that is indispensable for both macrocytosis and phagocytosis; DS primarily inhibits dynamin dependent endocytosis; and NS inhibits lipid rafts that play a role in caveolin-dependent endocytosis.

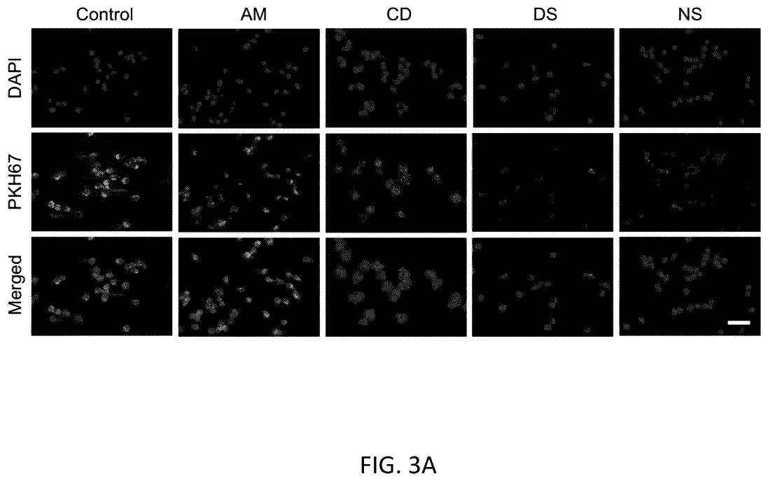

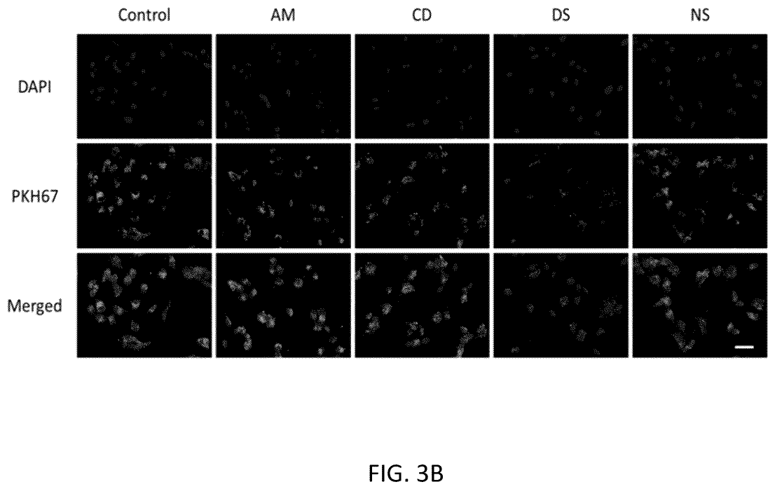

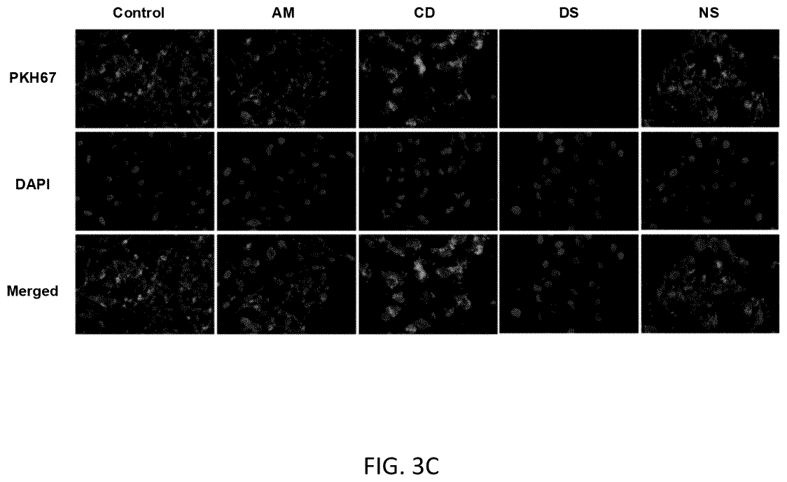

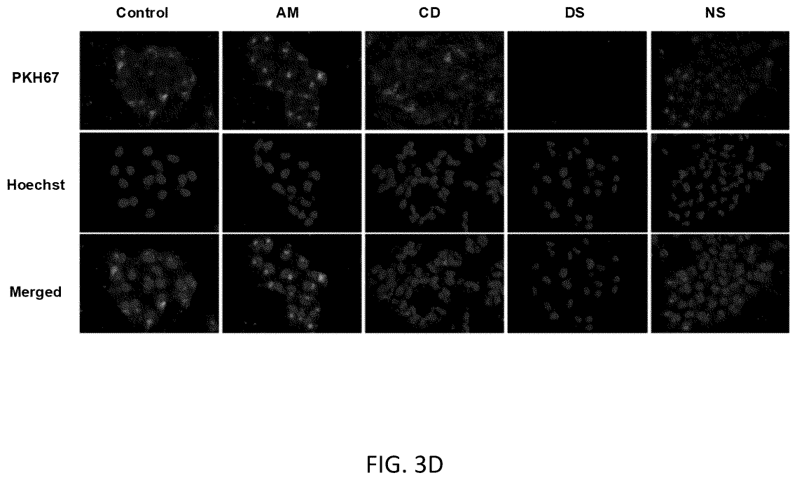

FIGS. 3A-3D depict the uptake of Bif.BEVs in various cell lines, including murine LL/2 (FIG. 3A), human A549 (FIG. 3B), Calu-1 (FIG. 3C), and H1792 cells (FIG. 3D). In some embodiments, the various cell lines may be pre-treated with endocytosis inhibitors for 2 hours and then with PKH67 stained 5 μg/mL Bif.BEVs for 24 hours. In some embodiments, the uptake of Bif.BEVs in LL/2 cells may be consistently and most prominently inhibited by DS, suggesting dynamin-dependent endocytosis represents one of the major mechanisms for cellular uptake of Bif.BEVs.

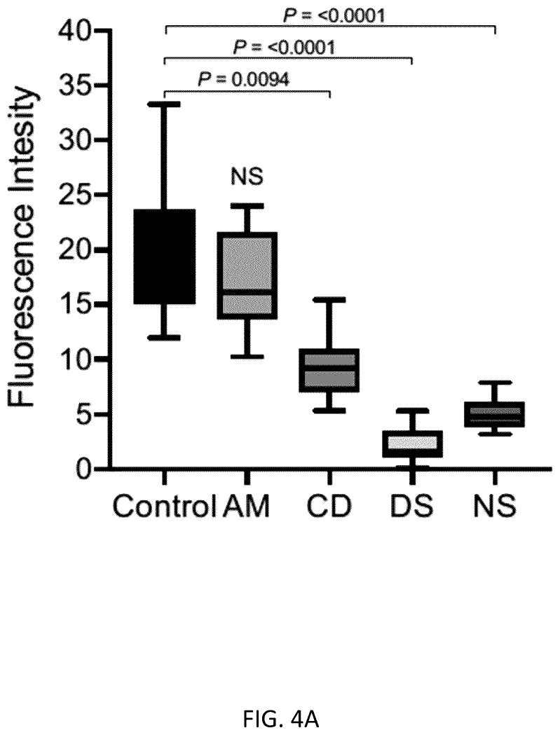

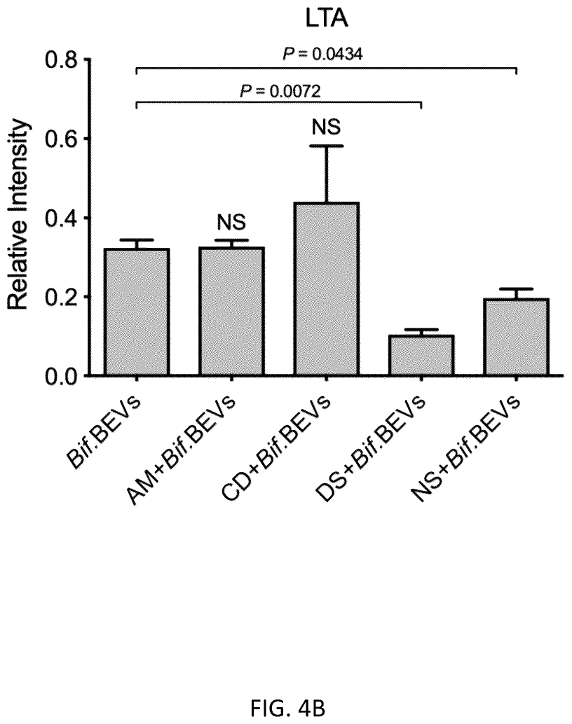

FIGS. 4A and 4B depict quantitative characterization data corresponding to Bif.BEVs. FIG. 4A shows the inhibition of Bif.BEV uptake upon treatment with DS. FIG. 4B shows the quantification of a western blot analysis of LTA data after LL/2 cells were pre-treated with endocytosis inhibitors for 2 hours and then with 5 μg/mL Bif.BEVs. In some embodiments, the expression of LTA may be lowest in DS treated cells, confirming that the uptake of Bif.BEVs may rely on dynamin-dependent endocytosis. In some embodiments, isogenic LL/2 cells with stable dynamin I/II knockdown may be generated using an shRNA approach.

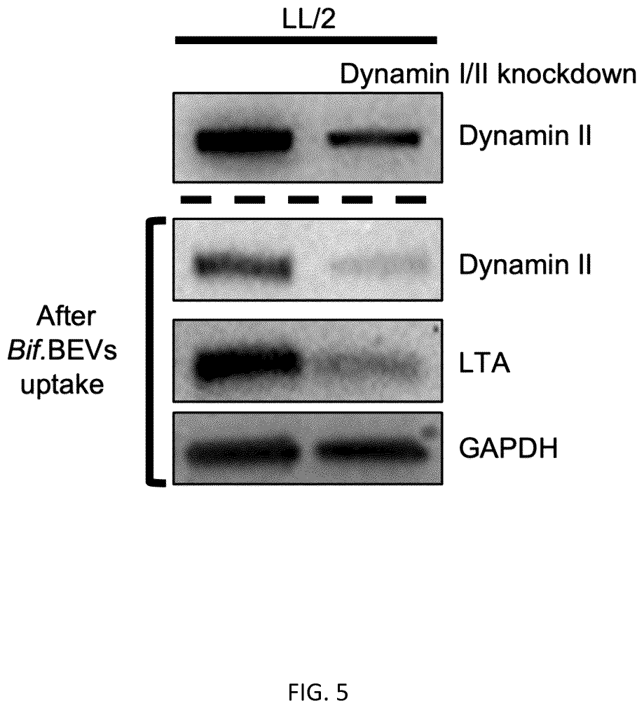

FIG. 5 includes data depicting that after incubating LL/2 cells with Bif.BEVs, a reduced detection of LTA in dynamin-deficient cells may be observed, which may indicate a decreased uptake of Bif.BEVs. Additionally, changes in genes associated with dynamin-dependent endocytosis, including dynamin I/II/III, clathrin, and EPS15 may be observed. For example, FIG. 5 depicts data indicating that exposure to Bif.BEVs in LL/2 cells may result in the upregulation of dynamin II/III, clathrin, and EPS15. These effects may be inhibited by DS, with the most notable reduction seen in dynamin expression. Together, these results suggest that dynamin may be used in the endocytosis of Bif.BEVs.

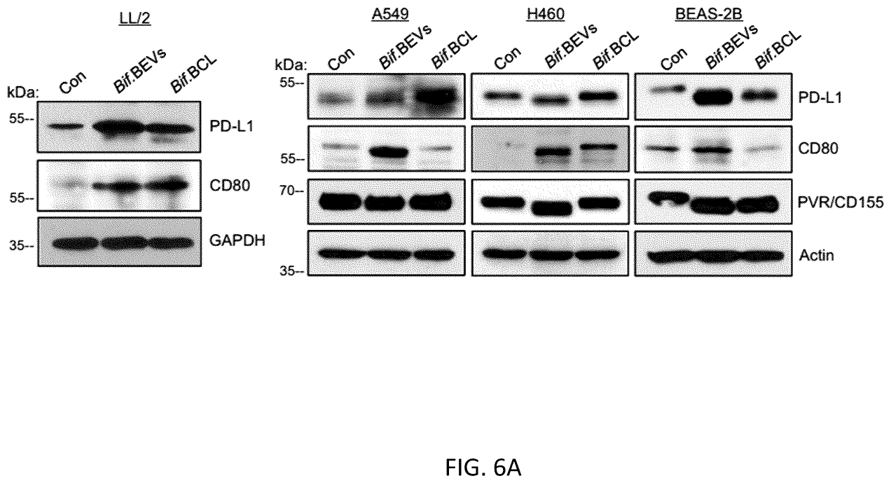

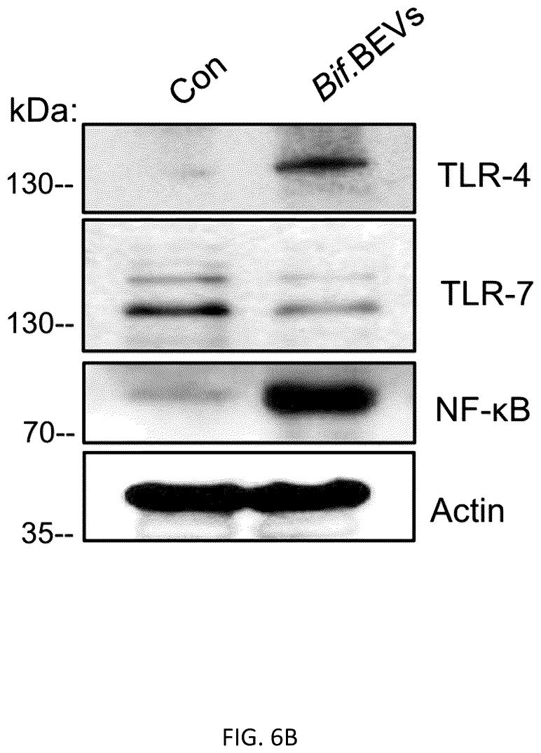

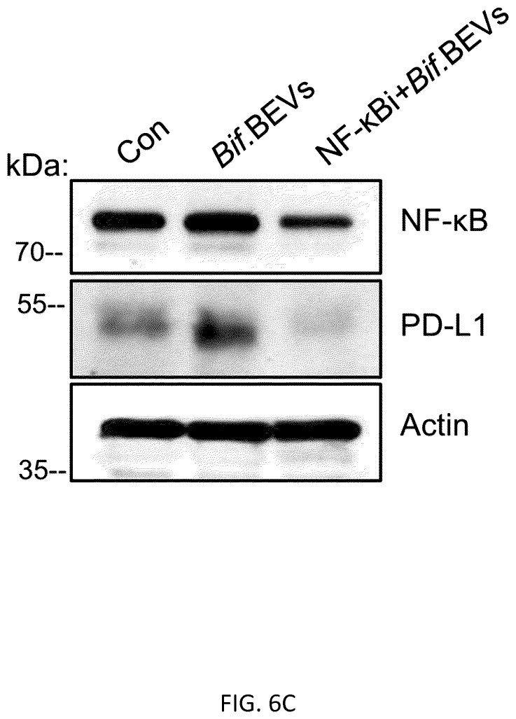

FIGS. 6A-6C depict additional quantitative characterization data corresponding to Bif.BEVs. To test whether Bif.BEVs may subsequently modulate the expression level of immune checkpoints (e.g., PD-L1), murine LL/2 and human lung cancer cells A549, H460 and lung immortal noncancerous cells BEAS-2B may be treated with Bif.BEVs at a concentration of 5 μg/mL Bif.BEVs for 48 hours (e.g., FIG. 6A). Using western blot analysis, the expression levels of immune checkpoints, including PD-L1, CD80 and PVR/CD155 may be examined. In some embodiments, Bif.BEVs may show significant and consistent increase in PD-L1 expression in human (A549, H460) and mouse (LL/2) lung cancer cells, as well as in immortalized non-tumorigenic lung BEAS-2B cells. In some embodiments, expression of CD80 in cell lines treated by Bif.BEVs may be increased. In some embodiments, Bif.B bacterial cell lysates (Bif.BCL) may be able to induce similar changes, suggesting possibly a shared component of Bif.BEVs and Bif.BCL may be responsible for this effect. To test whether LTA regulation of PD-L1 via the Toll-like receptor (TLR)-NF-κB pathway may be the mechanism of Bif.BEVs inducing PD-L1 expression, LL/2 cells may be treated with 5 μg/mL Bif.BEVs for 48 hours and western blot analysis of the levels of several TLRs (TLR-2, TLR-4, TLR-6 TLR-7) may be performed. In some embodiments, increased expression of both TLR-4 and NF-κB may be seen (e.g., FIG. 6B) when cells are treated with Bif.BEVs as compared to a control. In some embodiments, cells may be pre-treated with an NF-κB inhibitor for 2 hours and then treated with 5 μg/mL Bif.BEVs for 48 hours (e.g., FIG. 6C). In some embodiments, treatment with Bif.BEVs may lead to increased expression of NF-κB and PD-L1, which may be abolished after pre-treatment with the NF-κB inhibitor. In some embodiments, the mechanism of PD-L1 upregulation may result from dynamin-dependent endocytosis of Bif.BEVs activating the TLR-4-NF-κB pathway which then induce the expression of PD-L1.

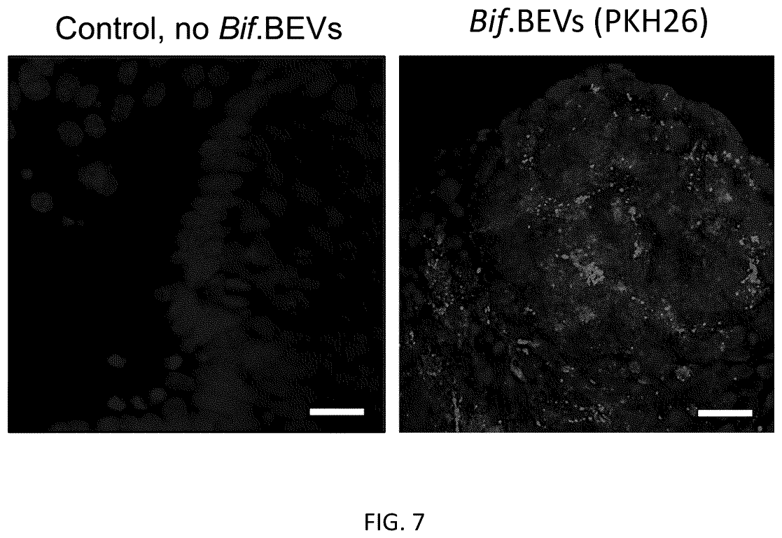

FIG. 7 depicts the uptake of Bif.BEVs by organoids. In some embodiments, the biodistribution of BEVs in organoids may be seen.

In some embodiments, examination of Bif.BEVs getting into and accumulating inside cells in a three-dimensional environment may be performed. In some embodiments, intestinal organoids may be developed by collecting the crypts from 15-week-old C57BL/6 mice and processing them to culture organoids. In these and other embodiments, intestinal organoids may grow into typical crypt-villus architecture and have regenerative capabilities in culture. After 7 days in culture, organoids may be treated with PKH26 (red) labeled Bif.BEVs (10 μg/mL) for 24 hours along with a no BEV control. After treatment, the organoids may be stained with 1 μg/mL of Hoechst for 30 min at 37° C. In some embodiments, confocal microscopy may show the presence of Bif.BEVs in both the intestinal cells and crypt lumen, therefore providing evidence of Bif.BEVs' internalization into the intestinal cells and penetration through into the lumen of crypts.

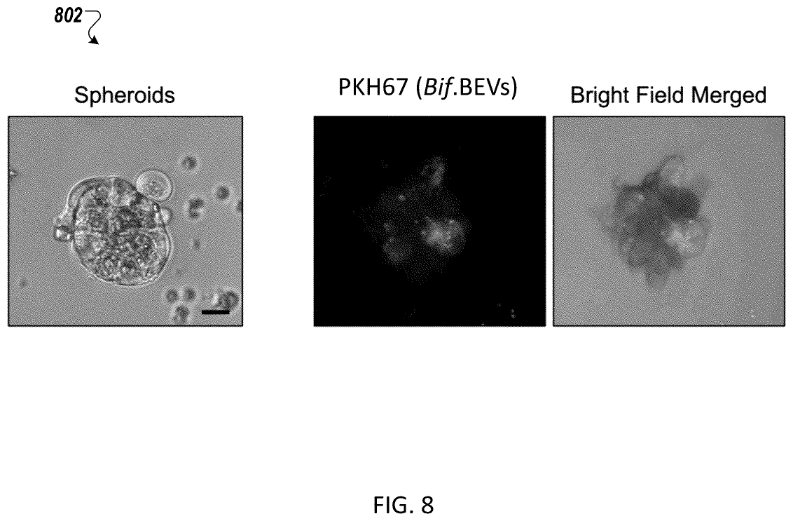

In some embodiments, the potential for Bif.BEVs to be taken up by patient-derived lung tumor spheroids or organoids may be evaluated. The bright field image 802 in FIG. 8 depicts a 14-day-old spheroid developed from human lung adenocarcinoma tissue. These floating spheroids may be treated with PKH67 (green) labeled extracellular vesicles (5 μg/mL) for 24 hours. Following treatment, confocal microscopy may reveal significant uptake of BEVs (green) in the spheroids.

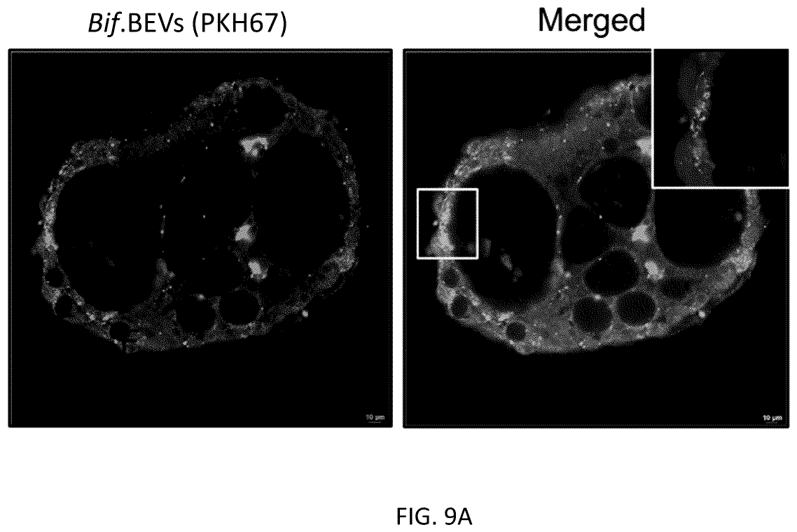

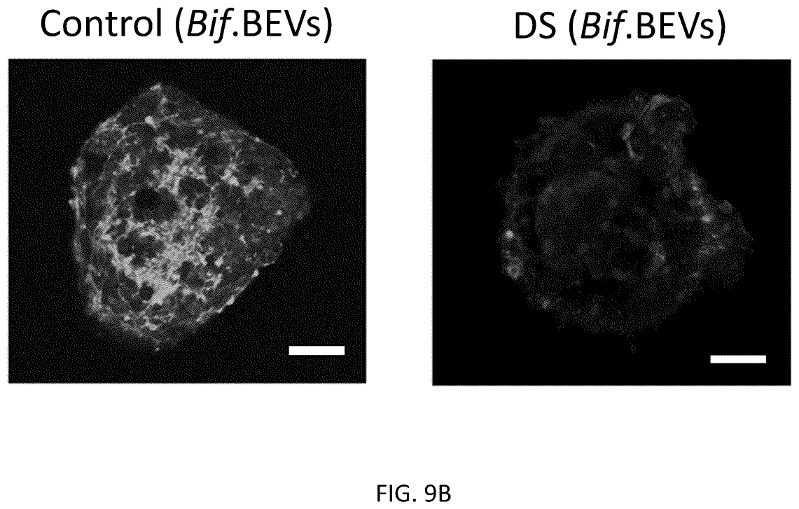

FIG. 9A depicts immunofluorescence imaging of organoids treated with PKH67-labeled Bif.BEVs (10 μg/mL) for 24 hours, which may indicate substantial uptake of Bif.BEVs and Bif.BEV presence in both the luminal side and interstitial regions.

FIG. 9B depicts lung adenocarcinoma organoids treated with DS inhibitor, resulting in the reduced uptake of PKH67-labeled Bif.BEVs.

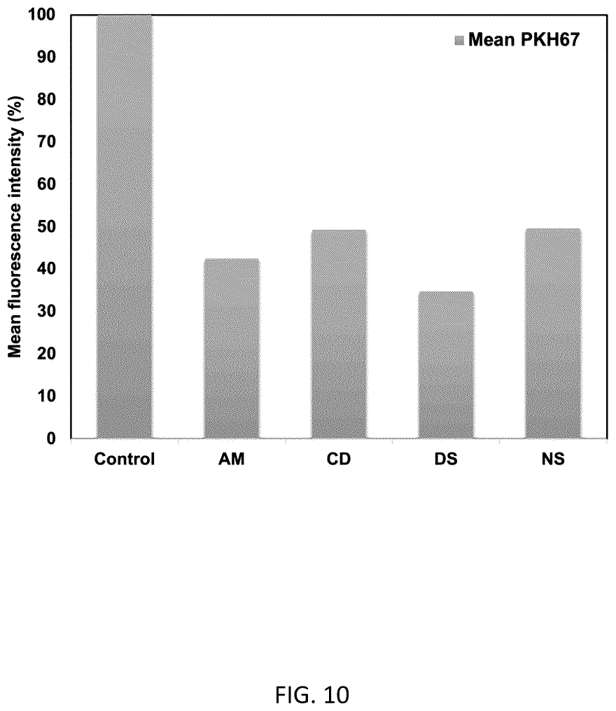

FIG. 10 depicts example evaluation data of endocytosis inhibitors on Bif.BEV update in three-dimensional organoids. In some embodiments, lung adenocarcinoma organoids may be treated with the immune checkpoint inhibitors. In these and other embodiments, the organoids may be dissociated into a single-cell suspension to quantify the uptake of Bif.BEVs via flow cytometry. Mean fluorescent intensity of cells exhibiting green fluorescence, indicating the uptake of PKH67-stained Bif.BEVs, may be measured. In some embodiments, consistent inhibition with various endocytosis inhibitors may be observed with the strongest effect being with DS use. However, unlike in a two-dimensional context, in some embodiments, significant inhibition with AM may also be observed, which may suggest that macropinocytosis may also play a significant role in Bif.BEVs' endocytosis.

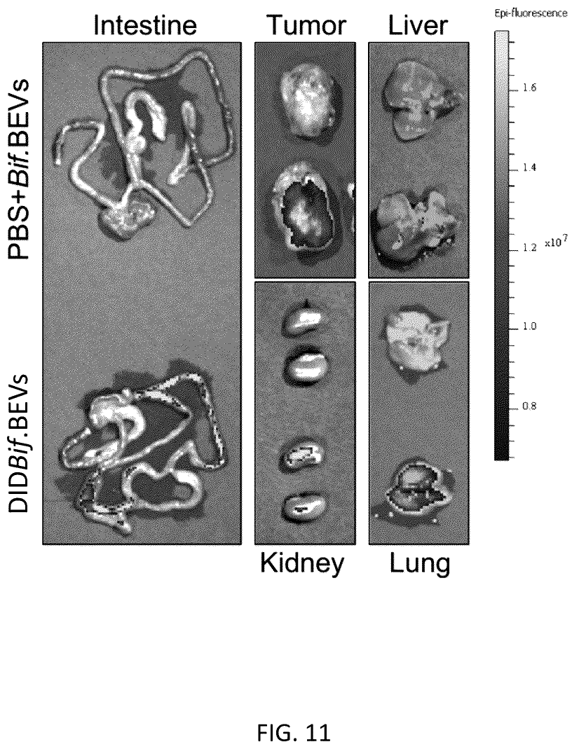

FIG. 11 depicts in vivo images of a syngeneic mouse tumor model after oral administration of Bif.BEVs. In some embodiments, a syngeneic tumor model may be established by injecting LL/2 cells subcutaneously into the flanks of syngeneic C57BL/6 mice. After tumors reach 1.5 cm³ in size, Bif.BEVs may be labeled with far-red fluorescent dye (e.g., DID [1,1-Dioctadecyl-3,3,3,3-tetramethylindodicarbocyanine]) and orally administered to the mice. In some embodiments, eight hours later, mice may be sacrificed, and various tissue and organs may be harvested and imaged using IVIS imaging. In some embodiments, the organs may be imaged side by side from mice treated with DID-labeled Bif.BEVs compared to PBS-dissolved Bif.BEVs to have a direct comparison. The radiant efficiency may be measured and a significant increase in far-red fluorescence may be observed in various organs (e.g., intestine, liver, kidney, lung) and tumors in mice treated with DID-labeled Bif.BEVs. In some embodiments, a strong radiant efficiency may be noted in tumors as shown by the quantification of tumors in a bar diagram (e.g., in FIG. 11), suggesting a significant distribution of Bif.BEVs inside of tumors. Additionally or alternatively, various degrees of Bif.BEVs accumulation may be observed in other organs, such as the intestine, lungs, kidney, liver, etc. In some embodiments, such biodistribution data may demonstrate that Bif.BEVs may cross the intestinal barrier and travel to distant tumor sites to potentially modulate a TME/TIME.

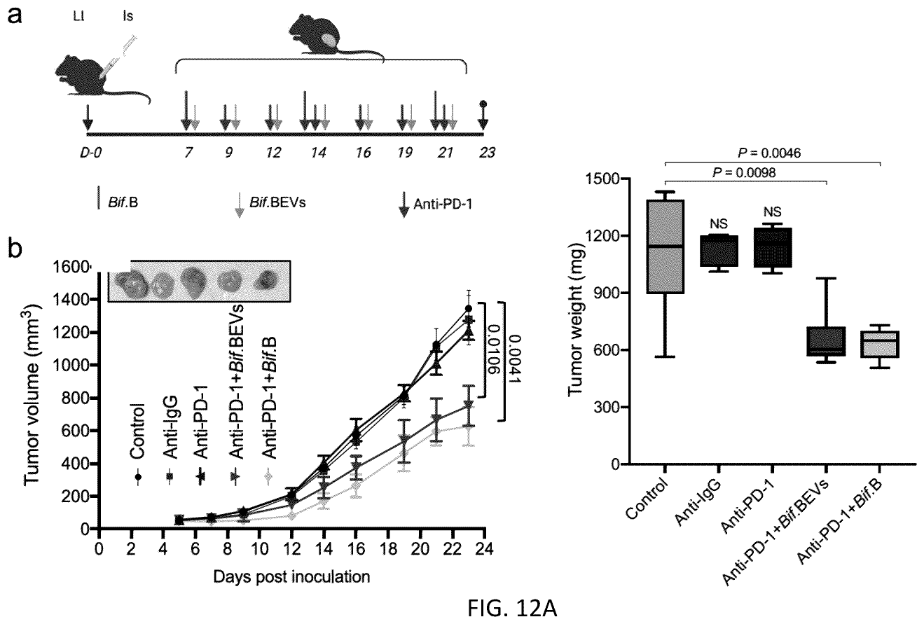

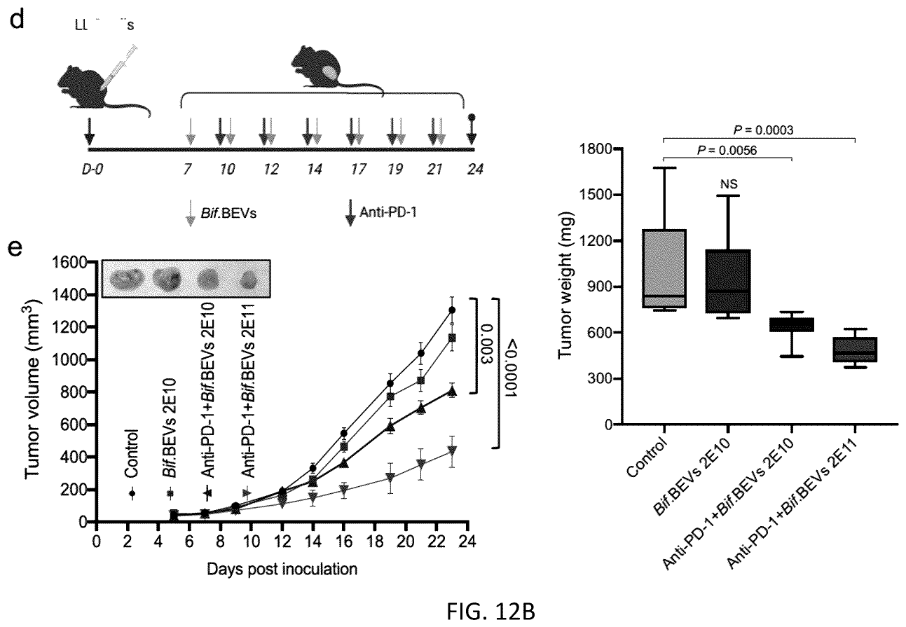

FIGS. 12A and 12B depict data indicating that Bif.BEVs enhance the therapeutic effect of anti-PD-1 in a syngeneic mouse model. In some embodiments, Bif.BEVs may enhance the therapeutic effect of anti-PD-1 in NSCLC. In some embodiments, LL/2 cells may be subcutaneously injected in both flanks of C57BL/6 mice. In these and other embodiments, mice may be subdivided into 5 groups: an untreated control (control), an IgG Control (IgG), an anti-PD-1 only (anti-PD-1), an anti-PD-1+Bif.BEVs (2×10¹⁰), and an anti-PD-1+Bif.B group. The tumors may be allowed to grow for a week and then treatment may be started. In some embodiments, when compared to the control group, both anti-IgG and anti-PD-1 groups may show similar growth without significant changes, which may confirm low efficacy of anti-PD-1 only in the LL/2 mouse model. In some embodiments, the anti-PD-1+Bif.BEVs (2×10¹⁰) and anti-PD-1+Bif.B groups showed significant decreases in tumor growth as compared to the control group. In these and other embodiments, the anti-PD-1+Bif.BEVs (2×10¹⁰) may show nearly comparable growth with the anti-PD-1+Bif.B group. In some embodiments, the tumor weights recapitulated the observations from tumor volume.

In some embodiments, to determine whether Bif.BEVs can have a dose dependent response towards tumor suppression, LL/2 tumors may be treated with a 10-fold higher amount of Bif.BEVs to see the effect. For example, mice may be divided into four groups as Control, Bif.BEVs (2×10¹⁰), anti-PD-1+Bif.BEVs (2×10¹⁰), and anti-PD-1+Bif.BEVs (2×10¹¹), and each group may be treated in a similar fashion. In some embodiments, a Bif.BEVs group may be included to determine whether the effect in the previous experiment may be primarily due to Bif.BEVs or to the combinatorial effect with anti-PD-1. In some embodiments, Bif.BEVs (2×10¹⁰) alone did not result in a significant decrease in tumor growth as compared to the control group. However, in these and other embodiments, the anti-PD-1+Bif.BEVs (2×10¹¹) group (10× more Bif.BEVs) in combination with anti-PD-1 may show significant reduction in tumor volume and weight as compared to the anti-PD-1+Bif.BEVs (2×10¹⁰) and control groups. Thus, in some embodiments, BEVs derived from commensal Bif.B may enhance the therapeutic efficacy of anti-PD-1 Bif.BEVs plus anti-PD-1 mitigated tumor growth via modulating cytokine levels.

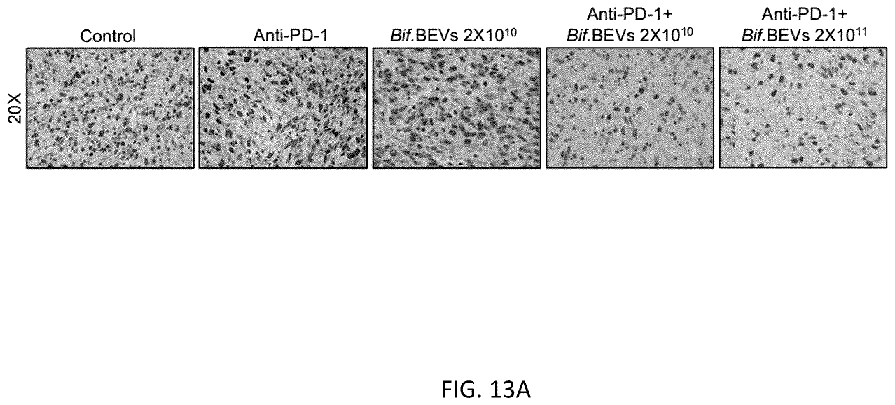

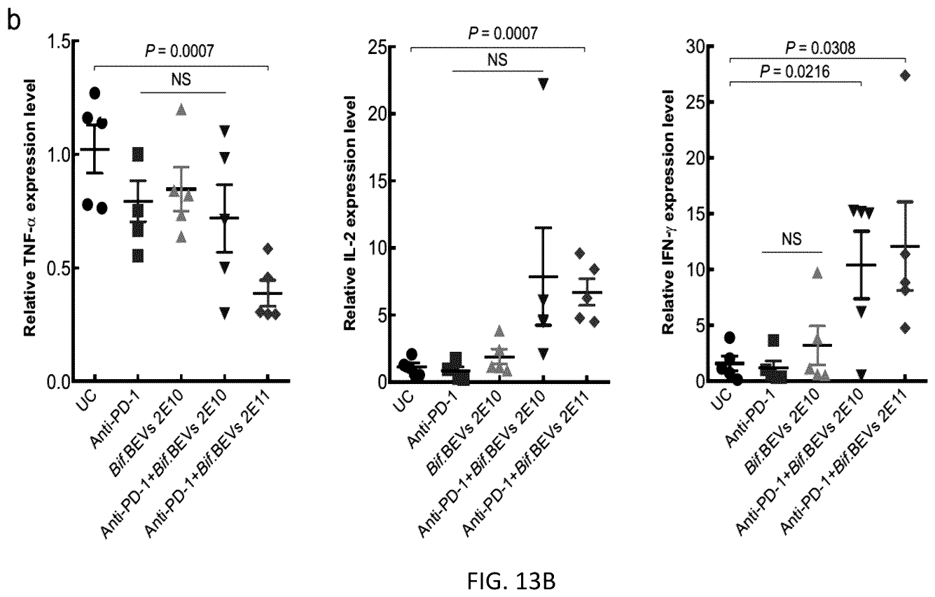

FIGS. 13A and 13B depict data indicating Bif.BEVs enhance anti-PD-1 efficacy via modulation of cytokines. To investigate how Bif.BEVs potentiate an anti-PD-1 effect, levels of Ki67, a reliable marker for tumor cell proliferation, may be measured. In some embodiments, the numbers of Ki67+ cells by IHC may be significantly lower in both the anti-PD-1+Bif.BEVs (2×10¹⁰) and anti-PD-1+Bif.BEVs (2×10¹¹) groups. In some embodiments, lower magnification images (10×) may be confirmatory of Bif.BEVs' capacity of potentiating the tumor suppressive effect of anti-PD-1 through reducing the proliferation of tumor cells.

Cytokine release may be a key element in determining the fate of tumor growth. Cytokines secreted by both the tumor and immune cells in the TIME may either induce or suppress tumor progression. In some embodiments, major cytokines (TNF-α, IL-2, IFN-γ and IL-10) may be measured by quantitative PCR to understand the mechanism of Bif.BEV-induced tumor suppression in combination with anti-PD-1 therapy. The relative expression levels may be measured in the tumor tissues from all the 5 groups including control, anti-PD-1, Bif.BEVs (2×10¹⁰), anti-PD-1+Bif.BEVs (2×10¹⁰), anti-PD-1+Bif.BEVs (2×10¹¹). In some embodiments, the level of proinflammatory cytokine TNF-α35, may be significantly decreased in anti-PD-1+BEVs (2×10¹¹) as compared to the control group. TNF-α signaling may impair the accumulation of CD8⁺ tumor-infiltrating T lymphocytes. IL-2 may play a role in the activation of immune cells. In some embodiments, the expression of IL-2 may be significantly increased in the anti-PD-1+Bif.BEVs (2×10¹¹) group, which may indicate the activation of T cells. In some embodiments, IFN-γ levels may be measured (e.g., because IFN-γ may play an important role in stimulation of antitumor immune response by coordinating both innate and adaptive immune responses). The proapoptotic cytokine IFN-γ may show a significant increase in both anti-PD-1+Bif.BEVs (2×10¹⁰) and anti-PD-1+Bif.BEVs (2×10¹¹) groups as compared to the control, therefore resulting in enhanced anti-tumor immunity. In some embodiments, there may be no statistical difference in IL-10 expression levels. In some embodiments, Bif.BEVs along with anti-PD-1 may mitigate tumor growth at least through the modulation of major cytokines directly involved in cellular apoptosis, inflammation, and/or immune cell activation.

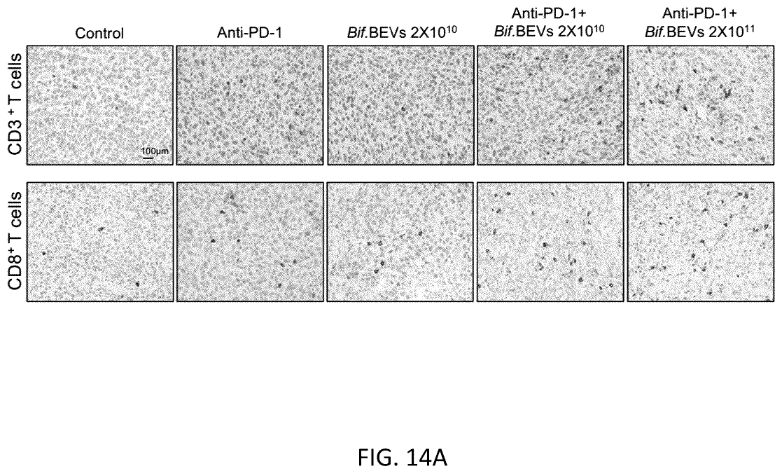

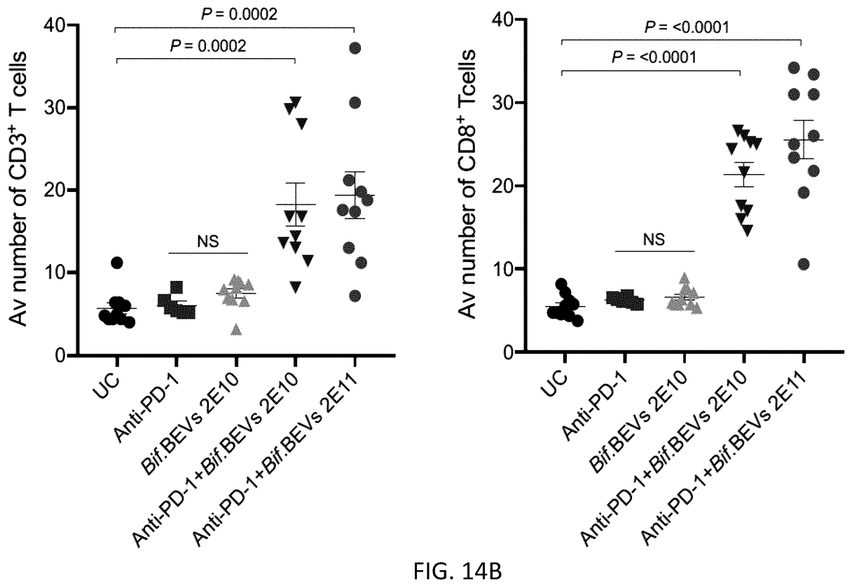

FIGS. 14A and 14B depict data indicating that Bif.BEVs in combination with anti-PD-1 increase tumor infiltrating T lymphocytes. In some embodiments, the numbers of CD3+ and CD8+ cells by IHC staining may be significantly higher in both anti-PD-1+Bif.BEVs (2×10¹⁰) and anti-PD-1+Bif.BEVs (2×10¹¹) groups while no significant changes may be noted in anti-PD-1 and Bif.BEVs alone groups when compared to the control group. In some embodiments, Bif.BEVs may potentiate anti-PD-1 through increasing tumor infiltrating cytotoxic T lymphocytes. In some embodiments, the effect of Bif.BEVs on an intratumoral Foxp3⁺ regulatory T-cell (Treg) population may be assessed via IHC in tumor tissue sections.

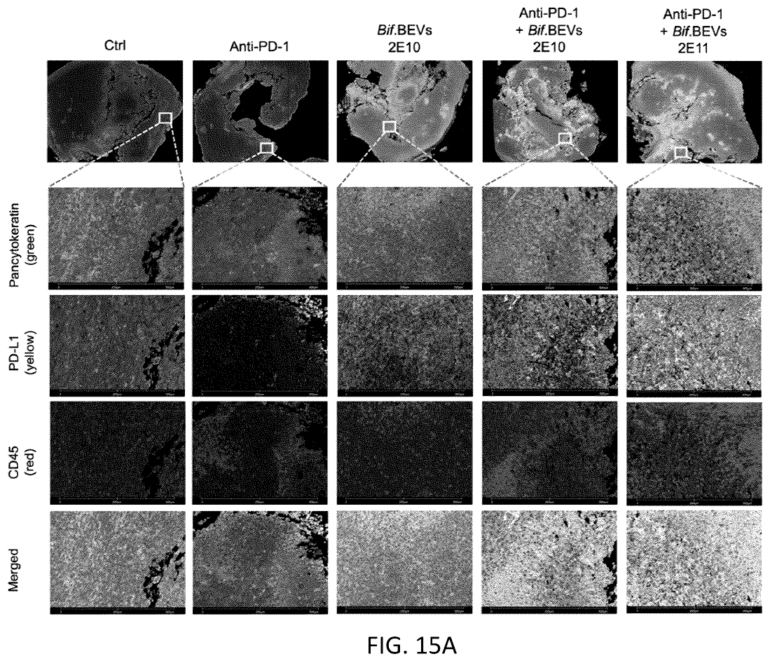

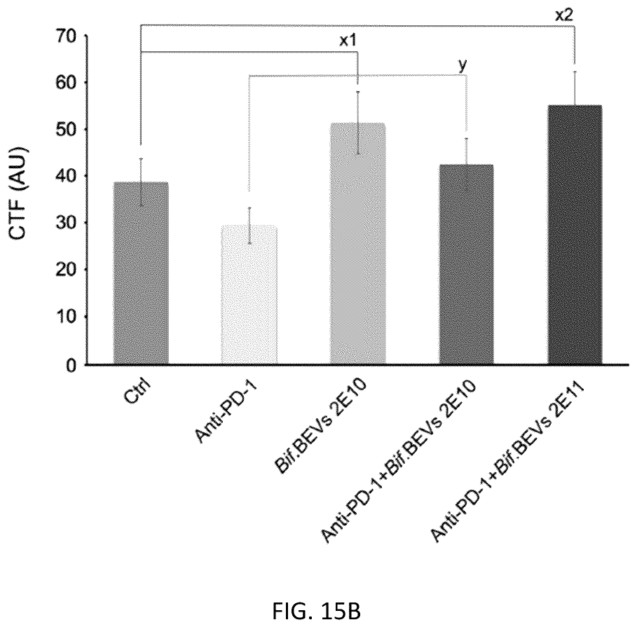

FIGS. 15A and 15B depict data indicating that Bif.BEVs upregulate PD-L1 expression in tumor cells and modulate immune and oncogenic pathways. In some embodiments, multiplexed immunofluorescence imaging may be combined using a digital spatial profiling (DSP) instrument with bulk RNA sequencing (RNAseq) to investigate the impact of Bif.BEVs on TME/TIME. In some embodiments, pancytokeratin staining (green) may be used to select areas with similar quantities of tumor cells for comparison, revealing that Bif.BEVs may significantly increase PD-L1 expression in these cells (yellow stained). In some embodiments, when combined with the anti-PD-1 agent, there may be a notable increase in tumor-infiltrating immune cells (stained red with an anti-CD45 antibody), which together may contribute to an enhanced anti-tumor effect (merged images).

In these and other embodiments, the increase in PD-L1 expression may be statistically significant in the Bif.BEVs (2×10¹⁰) and anti-PD-1+Bif.BEVs (2×10¹¹) groups compared to the control group (p=0.040 and 0.0047, respectively), but not in the anti-PD-1+Bif.BEVs (2×10¹⁰) group (p=0.382). In some embodiments, the observed PD-L1 upregulation may be offset by the killing of PD-L1 positive cancer cells following the addition of an anti-PD-1 agent. For example, in some embodiments, the anti-PD-1 group may exhibit lower PD-L1 expression than the control group, and the anti-PD-1+Bif.BEVs (2×10¹⁰) group may show significantly higher PD-L1 expression compared to the anti-PD-1 group (p=0.011).

In some embodiments, protein levels of cytokines TNF-α, IL-2, and IFN-γ may be analyzed. In these and other embodiments, the protein levels of these cytokines may generally correspond to their mRNA levels, which may confirm the impact of Bif.BEVs on cytokine production. Bif.BEVs alone increased IL-2 protein level. However, such an effect may be not significant at the mRNA level. IL-2 and IFN-γ staining signals correlated well with the CD45 signal, which may mark immune cells, suggesting these cytokines may be primarily produced by immune cells. In some embodiments, the correlation between TNF-α expression and the relative abundance of cancer versus immune cells may be less pronounced, suggesting that the contributions of immune and cancer cells to TNF-α production are not as markedly distinct as for IL-2 and IFN-γ.

In some embodiments, Bif.BEV contribution to immune boosting may be investigated by conducting bulk RNAseq on tumors from mice treated with either anti-PD-1 alone or anti-PD-1 plus Bif.BEVs. In these and other embodiments, substantial changes in gene expression may occur, leading to alterations in pathways relevant to immunotherapy response. For example, the upregulation of MHC peptide assembly and presentation, increased production or response to IFN-α or γ, enhanced T cell mediated cytotoxicity, and/or enhanced (i.e., reduced negative regulation of) toll-like receptor 4 (TLR4) signaling pathway may be observed. Additionally, significant downregulation of angiogenesis pathways may be observed. In some embodiments, Bif.BEVs may be found to downregulate several critical cancer growth pathways that contribute to immune suppression, such as Ras-MAPK and TGF-β signaling pathways.

### Pharmaceutical Compositions

Provided herein are compositions (e.g., pharmaceutical compositions) comprising BEV particles.

According to the present invention the BEV particles may be prepared as pharmaceutical compositions. It will be understood that such compositions can comprise one or more active ingredients, such as a BEV and a therapeutic agent, and, most often, a pharmaceutically acceptable excipient.

Relative amounts of the active ingredient (e.g. BEV particle and/or drug), a pharmaceutically acceptable excipient, and/or any additional ingredients in a pharmaceutical composition in accordance with the present disclosure may vary, depending upon the identity, size, and/or condition of the subject being treated and further depending upon the route by which the composition is to be administered. For example, the composition may comprise between 0.1% and 99% (w/v) of the active ingredient. By way of example, the composition may comprise between 0.1% and 100%, e.g., between 0.5 and 50%, between 1-30%, between 5-80%, at least 80% (w/w) active ingredient.

Although the descriptions of pharmaceutical compositions provided herein are principally directed to pharmaceutical compositions which are suitable for administration to humans, it will be understood by the skilled artisan that such compositions are generally suitable for administration to any other animal, e.g., to non-human animals, e.g. non-human mammals. Modification of pharmaceutical compositions suitable for administration to humans in order to render the compositions suitable for administration to various animals is well understood, and the ordinarily skilled veterinary pharmacologist can design and/or perform such modification with merely ordinary, if any, experimentation. Subjects to which administration of the pharmaceutical compositions is contemplated include, but are not limited to, humans and/or other primates; mammals, including commercially relevant mammals such as cattle, pigs, horses, sheep, cats, dogs, mice, rats, birds including commercially relevant birds such as poultry, chickens, ducks, geese, and/or turkeys.

In some embodiments, compositions are administered to humans, human patients or subjects.

### Formulations

Formulations of the pharmaceutical compositions described herein may be prepared by any method known or hereafter developed in the art of pharmacology. As used herein the term “pharmaceutical composition” refers to compositions comprising at least one active ingredient and optionally one or more pharmaceutically acceptable excipients.

In general excipient and/or one or more other accessory ingredients. As used herein, the phrase “active ingredient” generally refers either to an BEV particle with or without a drug molecule.

Formulations of the BEV particles and pharmaceutical compositions described herein may be prepared by any method known or hereafter developed in the art of pharmacology. In general, such preparatory methods include the step of bringing the active ingredient into association with an excipient and/or one or more other accessory ingredients, and then, if necessary and/or desirable, dividing, shaping and/or packaging the product into a desired single- or multi-dose unit.

A pharmaceutical composition in accordance with the present disclosure may be prepared, packaged, and/or sold in bulk, as a single unit dose, and/or as a plurality of single unit doses. As used herein, a “unit dose” refers to a discrete amount of the pharmaceutical composition comprising a predetermined amount of the active ingredient. The amount of the active ingredient is generally equal to the dosage of the active ingredient which would be administered to a subject and/or a convenient fraction of such a dosage such as, for example, one-half or one-third of such a dosage.

In some embodiments, a pharmaceutically acceptable excipient may be at least 95%, at least 96%, at least 97%, at least 98%, at least 99%, or 100% pure. In some embodiments, an excipient is approved for use for humans and for veterinary use. In some embodiments, an excipient may be approved by United States Food and Drug Administration. In some embodiments, an excipient may be of pharmaceutical grade. In some embodiments, an excipient may meet the standards of the United States Pharmacopoeia (USP), the European Pharmacopoeia (EP), the British Pharmacopoeia, and/or the International Pharmacopoeia.

Excipients, as used herein, include, but are not limited to, any and all solvents, dispersion media, diluents, or other liquid vehicles, dispersion or suspension aids, surface active agents, isotonic agents, thickening or emulsifying agents, preservatives, and the like, as suited to the particular dosage form desired. Various excipients for formulating pharmaceutical compositions and techniques for preparing the composition are known in the art (see Remington: The Science and Practice of Pharmacy, 21st Edition, A. R. Gennaro, Lippincott, Williams & Wilkins, Baltimore, Md., 2006; incorporated herein by reference in its entirety). The use of a conventional excipient medium may be contemplated within the scope of the present disclosure, except insofar as any conventional excipient medium may be incompatible with a substance or its derivatives, such as by producing any undesirable biological effect or otherwise interacting in a deleterious manner with any other component(s) of the pharmaceutical composition.

Exemplary diluents include, but are not limited to, calcium carbonate, sodium carbonate, calcium phosphate, dicalcium phosphate, calcium sulfate, calcium hydrogen phosphate, sodium phosphate lactose, sucrose, cellulose, microcrystalline cellulose, kaolin, mannitol, sorbitol, inositol, sodium chloride, dry starch, cornstarch, powdered sugar, etc., and/or combinations thereof.

In some embodiments, BEV particle formulations may comprise at least one inactive ingredient. As used herein, the term “inactive ingredient” refers to one or more agents that do not contribute to the activity of the active ingredient of the pharmaceutical composition included in formulations. In some embodiments, all, none or some of the inactive ingredients which may be used in the formulations of the present invention may be approved by the US Food and Drug Administration (FDA).

As used herein, the phrase “pharmaceutically acceptable route of administration” or “route of administration” includes, but is not limited to, oral, sublingual, buccal, nasal, inhalational (pulmonary), rectal, vaginal, topical, transdermal, ocular (ophthalmic), nasal, otic, parenteral, and mucosal routes. In some aspects, parenteral administration of the compounds is provided. The term “parenteral” as used herein includes subcutaneous, intravenous, intramuscular, and the like.

### Materials and Methods

In some embodiments, lung cancer cell lines (e.g., A549, H460, LL/2, etc.) may be used to evaluate BEVs as novel therapeutic agents for disease treatment (e.g., FIG. 6A).

In some embodiment, human lung tumor tissues may be used. In these and other embodiments, the tissues may be obtained from surgically resected lung specimens. In some embodiments, the tissues may be processed for the generation of lung cancer organoids (e.g., the lung cancer organoids depicted in FIGS. 9A and 9B).

In some embodiments, bacterial culture and isolation of BEVs may include using a probiotic Bif.B cocktail. For example, the cocktail may include a mixture of Bifidobacterium infantis, Bifidobacterium bifidum lactis, Bifidobacterium longum, Bifidobacterium bifidum, Bifidobacterium lactis, Bifidobacterium lactis, and/or Bifidobacterium breve. Example Bif.B are depicted in FIG. 1A.

In some embodiments, the uptake of bacterial extracellular vesicles may include seeding A549, H460, BEAS-2B, and/or LL/2 cells on coverslips in 6-well plates and allowing for attachment. The BEVs may be stained with either PKH67 (green) (e.g., FIG. 8) or PKH26 (red) (e.g., FIG. 7) fluorescent cell linkers. In some embodiments, the cells may be treated with 5 μg/mL of stained BEVs for 24 hours.

In some embodiments, organoid uptake may include seeding organoids in a solubilized basement membrane matrix until maturation on multiwell plates and then treating with Bif.BEVs (e.g., 10 μg/mL) stained with either PKH67 or PKH26 fluorescent cell linker for 24 hours. In these and other embodiments, organoids may be stained with 200 μL DAPI at a final concentration of 5 μg/mL for 20 min at 37° C. and then may be washed three times with PBS. In some embodiments, stained organoids may be imaged using a confocal microscope.

In some embodiments, endosomal uptake inhibition may include LL/2 and A549 cells (2×10⁵) being seeded in a 6-well plate with added coverslips into each well. in some embodiments, the cells may be pre-treated with the endosomal inhibitors Dynasore (DS), Cytochalasin D (CD), Nystatin (NS), Amiloride (AM), or a vehicle control (e.g., for 2 hours). The cells may be treated with 5 μg/mL Bif.BEVs stained with PKH67 (e.g., for 24 hours).

In some embodiments, Bif.BEVs may be quantified for their particle size and concentration by nanoparticle tracking analysis (NTA) (e.g., FIG. 1B).

In some embodiments, C57B L/6 J mice may be used to derive small intestinal organoid cultures. Small intestinal tissues (10 cm length cut into ˜2 mm pieces) may be collected in sterile PBS and washed several times with PBS supplemented with 2% penicillin-streptomycin. The crypts may be isolated. After dissociation of crypts, the supernatants may be passed through a cell strainer. Crypts in the filtrates may be pelleted by centrifugation and resuspended. In some embodiments, the crypt suspension may be incubated until gelification occurs. In some embodiments, growth medium may be added to support organoid development from seeded crypts. In these and other embodiments, organoids may be grown at 37° C. in 5% CO₂ incubator for 14 days.

For culturing patient-derived lung cancer organoids, lung tumor tissues (2-10 mm³) may be obtained from surgically resected specimens of NSCLC patients.

To study endocytosis in 3D organoid structures, organoids may be seeded and allowed to mature. After maturation, the organoids may be either untreated or pretreated with various endocytosis inhibitors, including AM, CD, DS, and NS, as above-mentioned, respectively. In some embodiments, PKH67-labeled Bif.BEVs (e.g., 10 μg/mL) may be added and the organoids may be incubated for 24 hours. In some embodiments, the uptake of Bif.BEVs may be quantified by flow cytometry by measuring the mean fluorescent intensity of cells exhibiting green fluorescence, indicating the uptake of PKH67-stained Bif.BEVs (e.g., FIGS. 2A-3D and 10).

In some embodiments, western blot analysis may be used (e.g., FIGS. 1D and 6A-6C). In these and other embodiments, western blot analysis may include separating lysates from BEVs and total cell lysates on SDS/PAGE gels and transferring to membranes. In some embodiments, the membranes may be blocked at room temperature with 10% milk for 2 hours, followed by overnight incubation with primary antibodies at 4° C. with gentle shaking. In some embodiments, western blot results may be quantified using the densitometric method.

In some embodiments, anti-PD-1 immune checkpoint inhibitors (e.g., agents) used may include nivolumab, pembrolizumab, cemiplimab, tislelizumab, dostarlimab, retifanlimab, and toripalimab. Additionally or alternatively, anti-PD-L1 immune check point inhibitors (e.g., agents) used may include atezolizumab, avelumab, durvalumab, and cosibelimab.

For the investigation of differential gene expression related to dynamin-dependent endocytosis, LL/2 cells (2×10⁵) may be seeded in a 6-well plate and experiments may be performed when cell confluency reaches 70-80%. Cells may be washed with PBS and pre-treated with DS, followed by treatment with 5 μg/mL Bif.BEVs, including control groups. After 24 hours of incubation, RNA may be extracted. In some embodiments, cDNA may be synthesized using total RNA. In these and other embodiments, RT-qPCR analysis may be performed. Additionally or alternatively, gene expression assays may be performed for the following targets: Dynamin I, Dynamin II, Dynamin III, Clathrin (Cltc), Epidermal growth factor receptor pathway substrate 15 (Eps15), PD-L1, and/or Glyceraldehyde-3-phosphate dehydrogenase (GAPDH) (e.g., FIG. 5). In some embodiments, mRNA expression levels may be quantified using the ΔΔCT method to calculate fold changes. For mouse tumor tissue studies, mouse tumor tissues may be used to extract the total RNA.

In some embodiments, immunohistochemistry may include fixing tumor tissues using a formalin-fixed, paraffin-embedded (FFPE) method and then cutting the tissue into 4 μM thin sections. In some embodiments, the tumor sections may be deparaffinized by immersion in Xylene and then rehydrated in a graded series of ethanol solutions. Tumor sections may be blocked in serum blocking solution. Sections may be stained for 1 hour at room temperature with a primary antibody (e.g., anti-CD38ε, anti-CD4, anti-CD8α, anti-Ki67, etc.) diluted in blocking solution. Sections may be stained with a conjugated goat anti-rabbit polymer horseradish peroxidase (HRP) secondary antibody. In some embodiments, the sections may be counterstained with hematoxylin and/or dehydrated in graded series of ethanol.

In some embodiments, immunofluorescence imaging and analysis may be performed using a digital spatial profiling (DSP) instrument. In these and other embodiments, one or more of the following primary antibodies may be used: anti-pancytokeratin, anti-CD45, anti-PD-L1, anti-TNF-α, anti-IL-2, and anti-IFN-γ. For unconjugated primary antibodies, a secondary antibody conjugated to a fluorescent dye may be applied. To visualize the cell nuclei, the slides may be counterstained with Syto-13. In some embodiments, pancytokeratin may be used to label cancer cells, while CD45 may be used to label immune cells. For quantifications (e.g., FIGS. 4A and 4B), such as for PD-L1, immunofluorescence images may be analyzed using imaging software. For instance, five regions of interest (ROIs) may be randomly selected from each stained slide of tumors harvested from each experimental group for analysis. An additional ROI of the same size may be placed outside of the tissue boundary for background correction. For each ROI, the corrected total fluorescence (CTF) may be calculated by subtracting the measured background integrated density from the integrated density and then normalizing to the area of the ROI, allowing for comparison of CTF across different stained slides.

In some embodiments, bulk RNA sequencing (RNAseq) and analysis may be used to study the impact of Bif.BEVs on anti-PD-1 treatment of tumor samples. In some embodiments, scanning electron microscopy (SEM) may be used.

In some embodiments, Bif.BEVs may be negatively stained using carbon filmed copper grids The carbon grids may be blot dried on filter paper in a dish and then air dried. The dried grids with Bif.BEVs may be photographed using a transmission electron microscope.

In some embodiments, immunogold labeling of Bif.BEVs may be performed with anti-LTA (e.g., FIG. 1C).

In some embodiments, C57BL/6 J mice may be used. In some embodiments, experiments may be performed on 8-10 weeks old female mice. In these and other embodiments, syngeneic tumors (e.g., FIG. 11) may be developed using LL/2 cells. For example, subcutaneous injections may be done on both flanks of mice using LL/2 cell suspension and tumors may be allowed to grow (e.g., FIGS. 12A and 12B). Mice in a first group may be orally gavaged with Bif.BEVs three times a week. Mice in a second group may be orally gavaged with Bif.B in PBS. These two groups may be also injected with either anti-IgG or anti-PD-1. After the tumors reach 1.5 cm diameter in the control group, the mice may be sacrificed to collect the tumors. The formula used to calculate tumor volume may be V=(W²×L)/2, where W is tumor width and L is tumor length. Additionally or alternatively, anti-PD-1 doses may be given to the respective groups. Tumors may be measured, resected, and stored as per experimental needs.

With respect to the use of substantially any plural and/or singular terms herein, those having skill in the art can translate from the plural to the singular and/or from the singular to the plural as is appropriate to the context and/or application. The various singular/plural permutations may be expressly set forth herein for sake of clarity.

It will be understood by those within the art that, in general, terms used herein, and especially in the appended claims (e.g., bodies of the appended claims) are generally intended as “open” terms (e.g., the term “including” should be interpreted as “including but not limited to,” the term “having” should be interpreted as “having at least,” the term “includes” should be interpreted as “includes but is not limited to,” etc.). It will be further understood by those within the art that if a specific number of an introduced claim recitation is intended, such an intent will be explicitly recited in the claim, and in the absence of such recitation, no such intent is present. For example, as an aid to understanding, the following appended claims may contain usage of the introductory phrases “at least one” and “one or more” to introduce claim recitations. However, the use of such phrases should not be construed to imply that the introduction of a claim recitation by the indefinite articles “a” or “an” limits any particular claim containing such introduced claim recitation to embodiments containing only one such recitation, even when the same claim includes the introductory phrases “one or more” or “at least one” and indefinite articles such as “a” or “an” (e.g., “a” and/or “an” should be interpreted to mean “at least one” or “one or more”); the same holds true for the use of definite articles used to introduce claim recitations. In addition, even if a specific number of an introduced claim recitation is explicitly recited, those skilled in the art will recognize that such recitation should be interpreted to mean at least the recited number (e.g., the bare recitation of “two recitations,” without other modifiers, means at least two recitations, or two or more recitations). Furthermore, in those instances where a convention analogous to “at least one of A, B, and C, etc.” is used, in general, such a construction is intended in the sense one having skill in the art would understand the convention (e.g., “a system having at least one of A, B, and C” would include but not be limited to systems that have A alone, B alone, C alone, A and B together, A and C together, B and C together, and/or A, B, and C together, etc.). In those instances where a convention analogous to “at least one of A, B, or C, etc.” is used, in general, such a construction is intended in the sense one having skill in the art would understand the convention (e.g., “a system having at least one of A, B, or C” would include but not be limited to systems that have A alone, B alone, C alone, A and B together, A and C together, B and C together, and/or A, B, and C together, etc.). It will be further understood by those within the art that virtually any disjunctive word and/or phrase presenting two or more alternative terms, whether in the description, claims, or drawings, should be understood to contemplate the possibilities of including one of the terms, either of the terms, or both terms. For example, the phrase “A or B” will be understood to include the possibilities of “A” or “B” or “A and B.”

In addition, where features or aspects of the disclosure are described in terms of Markush groups, those skilled in the art will recognize that the disclosure is also thereby described in terms of any individual member or subgroup of members of the Markush group.

As will be understood by one skilled in the art, for any and all purposes, such as in terms of providing a written description, all ranges disclosed herein also encompass any and all possible subranges and combinations of subranges thereof. Any listed range can be easily recognized as sufficiently describing and enabling the same range being broken down into at least equal halves, thirds, quarters, fifths, tenths, etc. As a non-limiting example, each range discussed herein can be readily broken down into a lower third, middle third and upper third, etc. As will also be understood by one skilled in the art all language such as “up to,” “at least,” and the like include the number recited and refer to ranges which can be subsequently broken down into subranges as discussed above. Finally, as will be understood by one skilled in the art, a range includes each individual member. Thus, for example, a group having 1-3 cells refers to groups having 1, 2, or 3 cells. Similarly, a group having 1-5 cells refers to groups having 1, 2, 3, 4, or 5 cells, and so forth.

From the foregoing, it will be appreciated that various embodiments of the present disclosure have been described herein for purposes of illustration, and that various modifications may be made without departing from the scope and spirit of the present disclosure. Accordingly, the various embodiments disclosed herein are not intended to be limiting, with the true scope and spirit being indicated by the following claims.

All references recited herein are incorporated herein by specific reference in their entirety.
Gandhi, L. et al. Pembrolizumab plus chemotherapy in metastatic non-small-cell lung cancer. N. Engl. J. Med 378, 2078-2092 (2018).
Paz-Ares. L. et al. Pembrolizumab plus chemotherapy for squamous non-small-cell lung cancer. N. Engl. J. Med 379, 2040-2051 (2018).
Hellmann, M. D. et al. Nivolumab plus ipilimumab in advanced non-small-cell lung cancer. N. Engl. J. Med 381, 2020-2031 (2019).
Sezer, A. et al. Cemiplimab monotherapy for first-line treatment of advanced non-small-cell lung cancer with PD-L1 of at least 50%: a multicentre, open-label, global, phase 3, randomised, controlled trial. Lancet 397, 592-604 (2021).
Strouse, C., Mangalam, A. & Zhang. J. Bugs in the system: bringing the human microbiome to bear in cancer immunotherapy. Gut Microbes, 1-4 (2018).
Chau, J. & Zhang, J. Tying small changes to large outcomes: The cautious promise in incorporating the microbiome into immu-notherapy. Int. J. Mol. Sci. 22 (2021).
Sivan, A. et al. Commensal Bifidobacterium promotes antitumor immunity and facilitates anti-PD-L1 efficacy. Science 350, 1084-1089 (2015).
Wang, F., Yin, Q., Chen, L. & Davis, M. M. Bifidobacterium can mitigate intestinal immunopathology in the context of CTLA-4 blockade. Proc. Natl Acad. Sci. USA 115, 157-161 (2018).
Lee. S. H. et al. Bifidobacterium bifidum strains synergize with immune checkpoint inhibitors to reduce tumour burden in mice. Nat. Microbiol 6, 277-288 (2021).
Chau, J. et al. Prospective correlation between the patient micro-biome with response to and development of immune-mediated adverse effects to immunotherapy in lung cancer. BMC Cancer 21, 808 (2021).
Sun, S. et al. Bifidobacterium alters the gut microbiota and mod-ulates the functional metabolism of T regulatory cells in the context of immune checkpoint blockade. Proc. Natl Acad. Sci. USA 117, 27509-27515 (2020).
Binnewies, M. et al. Understanding the tumor immune micro-environment (TIME) for effective therapy. Nat. Med 24, 541-550 (2018).
Ross, T. J. & Zhang, J. The Microbiome-TIME Axis: A Host of Possi-bilities. Microorganisms 11 (2023).
van Niel, G., D'Angelo, G. & Raposo, G. Shedding light on the cell biology of extracellular vesicles. Nat. Rev. Mol. Cell Biol. 19, 213-228 (2018).
Hughes, D. T. & Sperandio, V. Inter-kingdom signalling: commu-nication between bacteria and their hosts. Nat. Rev. Microbiol 6, 111-120 (2008).
Tulkens, J., De Wever, O. & Hendrix, A. Analyzing bacterial extra-cellular vesicles in human body fluids by orthogonal biophysical separation and biochemical characterization. Nat. Protoc. 15, 40-67 (2020).
Chronopoulos, A. & Kalluri, R. Emerging role of bacterial extra-cellular vesicles in cancer. Oncogene 39, 6951-6960 (2020).
Kim, O. Y. et al. Bacterial outer membrane vesicles suppress tumor by interferon-gamma-mediated antitumor response. Nat. Commun. 8, 626 (2017).
Jones, E. J. et al. The uptake, trafficking, and biodistribution of bacteroides thetaiotaomicron generated outer membrane vesicles. Front Microbiol 11, 57 (2020).
Yaghoubfar, R. et al. Effect of Akkermansia muciniphila, faecali-bacterium prausnitzii, and their extracellular vesicles on the ser-otonin system in intestinal epithelial cells. Probiotics Antimicrob. Proteins 13, 1546-1556 (2021).
Nahui Palomino, R. A., Vanpouille, C., Costantini, P. E. & Margolis, L. Microbiota-host communications: Bacterial extracellular vesicles as a common language. PLOS Pathog. 17, e1009508 (2021).
Zhang, P. & Liu, Z. Structural insights into the transporting and catalyzing mechanism of DIB in LTA D-alanylation. Nat. Commun. 15, 3404 (2024).
Welsh, J. A. et al. Minimal information for studies of extracellular vesicles (MISEV2023): From basic to advanced approaches. J. Extracell. Vesicles 13, e12404 (2024).
Rennick, J. J., Johnston, A. P. R. & Parton, R. G. Key principles and methods for studying the endocytosis of biological and nanoparticle therapeutics. Nat. Nanotechnol. 16, 266-276 (2021).
Cecil, J. D. et al. Differential responses of pattern recognition receptors to outer membrane vesicles of three periodontal patho-gens. PLOS One 11, e0151967 (2016).
Wang, Y. et al. Adjuvant effect of the novel TLR1/TLR2 agonist diprovocim synergizes with anti-PD-L1 to eliminate melanoma in mice. Proc. Natl Acad. Sci. USA 115, E8698-E8706 (2018).
Ruiz, P. A., Hoffmann, M., Szcesny, S., Blaut, M. & Haller, D. Innate mechanisms for Bifidobacterium lactis to activate transient pro-inflammatory host responses in intestinal epithelial cells after the colonization of germ-free rats. Immunology 115, 441-450 (2005).
Lambies, G. et al. Cell polarity proteins promote macropinocytosis in response to metabolic stress. bioRxiv (2024).
Lechner, M. G. et al. Immunogenicity of murine solid tumor models as a defining feature of in vivo behavior and response to immu-notherapy. J. Immunother. 36, 477-489 (2013).
Mosely, S. I. et al. Rational selection of syngeneic preclinical tumor models for immunotherapeutic drug discovery. Cancer Immunol. Res 5, 29-41 (2017).
Graefe, C. et al. Optimized Ki-67 staining in murine cells: a tool to determine cell proliferation. Mol. Biol. Rep. 46, 4631-4643 (2019).
Tan, P. H. et al. Immunohistochemical detection of Ki67 in breast cancer correlates with transcriptional regulation of genes related to apoptosis and cell death. Mod. Pathol. 18, 374-381 (2005).
Setrerrahmane, S. & Xu, H. Tumor-related interleukins: old vali-dated targets for new anti-cancer drug development. Mol. Cancer 16, 153 (2017).
Perez-Ruiz, E. et al. Prophylactic TNF blockade uncouples efficacy and toxicity in dual CTLA-4 and PD-1 immunotherapy. Nature 569, 428-432 (2019).
Berraondo, P. et al. Cytokines in clinical cancer immunotherapy. Br. J. Cancer 120, 6-15 (2019).
Bertrand, F. et al. TNFalpha blockade overcomes resistance to anti-PD-1 in experimental melanoma. Nat. Commun. 8, 2256 (2017).
Jiang, T., Zhou, C. & Ren, S. Role of IL-2 in cancer immunotherapy. Oncoimmunology 5, e1163462 (2016).
Zhao, S. et al. Low-dose apatinib optimizes tumor microenviron-ment and potentiates antitumor effect of PD-1/PD-L1 blockade in lung cancer. Cancer Immunol. Res 7, 630-643 (2019).
Paijens, S. T., Vledder, A., de Bruyn, M. & Nijman, H. W. Tumor-infiltrating lymphocytes in the immunotherapy era. Cell Mol. Immunol. 18, 842-859 (2021).
Hou, K. et al. Microbiota in health and diseases. Signal Transduct. Target Ther. 7, 135 (2022).
Li, W. Z., Stirling, K., Yang, J. J. & Zhang, L. Gut microbiota and diabetes: From correlation to causality and mechanism. World J. Diab. 11, 293-308 (2020).
Tang, W. H., Kitai, T. & Hazen, S. L. Gut microbiota in cardiovascular health and disease. Circ. Res 120, 1183-1196 (2017).
Huang, C. et al. Relating gut microbiome and its modulating factors to immunotherapy in solid tumors: A systematic review. Front Oncol. 11, 642110 (2021).
Liu, B., Chau, J., Dai, Q., Zhong, C. & Zhang, J. Exploring gut microbiome in predicting the efficacy of immunotherapy in non-small cell lung cancer. Cancers (Basel) 14 (2022).
Baruch, E. N. et al. Fecal microbiota transplant promotes response in immunotherapy-refractory melanoma patients. Science 371, 602-609 (2021).
Bittel, M. et al. Visualizing transfer of microbial biomolecules by outer membrane vesicles in microbe-host-communication in vivo. J. Extracell. Vesicles 10, e12159 (2021).
Brown, L., Wolf, J. M., Prados-Rosales, R.& Casadevall, A. Through the wall: extracellular vesicles in Gram-positive bacteria, myco-bacteria and fungi. Nat. Rev. Microbiol 13, 620-630 (2015).
Johnston, E. L., Heras, B., Kufer, T. A. & Kaparakis-Liaskos, M. Detection of bacterial membrane vesicles by NOD-like receptors. Int. J. Mol. Sci. 22 (2021).
Kuehn, M. J. & Kesty, N. C. Bacterial outer membrane vesicles and the host-pathogen interaction. Genes Dev. 19, 2645-2655 (2005).
Schwechheimer, C. & Kuchn, M. J. Outer-membrane vesicles from Gram-negative bacteria: biogenesis and functions. Nat. Rev. Microbiol 13, 605-619 (2015).
Buzas, E. I. The roles of extracellular vesicles in the immune system. Nat. Rev. Immunol. 23, 236-250 (2023).
Toyofuku, M., Nomura, N. & Eberl, L. Types and origins of bacterial membrane vesicles. Nat. Rev. Microbiol 17, 13-24 (2019).
Zhang. B. & Perez, C. Stabilization and crystallization of a membrane protein involved in lipid transport. Methods Mol. Biol. 2127, 283-292 (2020).
Hesser, A. R., Schaefer, K., Lee, W. & Walker, S. Lipoteichoic acid polymer length is determined by competition between free starter units. Proc. Natl Acad. Sci. USA 117, 29669-29676 (2020).
Mulcahy, L. A., Pink, R. C. & Carter, D. R. Routes and mechanisms of extracellular vesicle uptake. J. Extracell Vesicles 3 (2014).
Antonangeli, F. et al. Regulation of PD-L1 Expression by NF-kappaB in Cancer. Front Immunol. 11, 584626 (2020).
Alberro, A., Iparraguirre, L., Fernandes, A. & Otaegui, D. Extra-cellular vesicles in blood: sources, effects, and applications. Int. J. Mol. Sci. 22 (2021).
Namork, E. & Brandtzaeg, P. Fatal meningococcal septicaemia with “blebbing” meningococcus. Lancet 360, 1741 (2002).
Tulkens, J. et al. Increased levels of systemic LPS-positive bacterial extracellular vesicles in patients with intestinal barrier dysfunction. Gut 69, 191-193 (2020).
Jeong, J. H. et al. Differential immune-stimulatory effects of LTAs from different lactic acid bacteria via MAPK signaling pathway in RAW 264.7 cells. Immunobiology 220, 460-466 (2015).
Weill, F. S. et al. Lipoteichoic acid from Lactobacillus rhamnosus GG as an oral photoprotective agent against UV-induced carcinogenesis. Br. J. Nutr. 109, 457-466 (2013).
Khan, M. W. et al. Modulating intestinal immune responses by lipoteichoic acid-deficient Lactobacillus acidophilus. Immunotherapy 4, 151-161 (2012).
Kalaora, S. et al. Identification of bacteria-derived HLA-bound peptides in melanoma. Nature 592, 138-143 (2021).
Pardoll, D. M. The blockade of immune checkpoints in cancer immunotherapy. Nat. Rev. Cancer 12, 252-264 (2012).
Seo, Y. D. et al. Toll-Like Receptor 4 agonist injection with con-current radiotherapy in patients with metastatic soft tissue sarcoma: A Phase 1 Nonrandomized Controlled Trial. JAMA Oncol. 9, 1660-1668 (2023).
Farias, A. et al. A TLR4 agonist improves immune checkpoint blockade treatment by increasing the ratio of effector to regulatory cells within the tumor microenvironment. Sci. Rep. 11, 15406 (2021).
Nelson, M. H. et al. Toll-like receptor agonist therapy can profoundly augment the antitumor activity of adoptively transferred CD8 (+) T cells without host preconditioning. J. Immunother. Cancer 4, 6 (2016).
Xu, D. et al. Feasibility between bifidobacteria targeting and changes in the acoustic environment of tumor tissue for synergistic HIFU. Sci. Rep. 10, 7772 (2020).
Skoulidis, F. et al. STK11/LKB1 Mutations and PD-1 Inhibitor Resistance in KRAS-mutant lung adenocarcinoma. Cancer Discov. 8, 822-835 (2018).
Zhao, Y. et al. PD-L1: CD80 Cis-heterodimer triggers the co-stimulatory receptor cd28 while repressing the inhibitory PD-1 and CTLA-4 Pathways. Immunity 51, 1059-1073.e 1059 (2019).
Sugiura, D. et al. Restriction of PD-1 function by cis-PD-L1/CD80 interactions is required for optimal T cell responses. Science 364, 558-566 (2019).
Park, J. S. et al. Targeting PD-L2-RGMb overcomes microbiome-related immunotherapy resistance. Nature 617, 377-385 (2023).
Park, J. S., Gazzaniga, F. S., Kasper, D. L.& Sharpe, A. H. Microbiota-dependent regulation of costimulatory and coinhibitory pathways via innate immune sensors and implications for immunotherapy. Exp. Mol. Med. 55, 1913-1921 (2023).
Xiao, H., Ye, X., Vishwakarma, V., Preet, R. & Dixon, D. A. CRC-derived exosomes containing the RNA binding protein HuR pro-mote lung cell proliferation by stabilizing c-Myc mRNA. Cancer Biol. Ther. 23, 139-149 (2022).
Kim, M. et al. Patient-derived lung cancer organoids as in vitro cancer models for therapeutic screening. Nat. Commun. 10, 3991 (2019).
Bolger, A. M., Lohse, M. & Usadel, B. Trimmomatic: a flexible trim-mer for Illumina sequence data. Bioinformatics 30, 2114-2120 (2014).
Dobin, A. et al. STAR: ultrafast universal RNA-seq aligner. Bioinfor-matics 29, 15-21 (2013).
Liao, Y., Smyth, G. K. & Shi, W. The R package Rsubread is easier, faster, cheaper and better for alignment and quantification of RNA sequencing reads. Nucleic Acids Res. 47, e47 (2019).
Love, M. I., Huber, W. & Anders, S. Moderated estimation of fold change and dispersion for RNA-seq data with DESeq2. Genome Biol. 15, 550 (2014).
Subramanian, A. et al. Gene set enrichment analysis: a knowledge-based approach for interpreting genome-wide expression profiles. Proc. Natl Acad. Sci. USA 102, 15545-15550 (2005).
Harris, M. A. et al. The gene ontology (GO) database and informatics resource. Nucleic Acids Res. 32, D258-D261 (2004).
Wu, T. et al. clusterProfiler 4.0: A universal enrichment tool for interpreting omics data. Innov. (Camb.) 2, 100141 (2021).
Vishwakarma, V. (2025). Schematic representation showing the timeline of tumor cell implantation, tumor formation and treatment with either Bif.BEVs followed by anti-PD-1 or isotype control. Created in BioRender.
Vishwakarma, V. (2025). Schematic representation of a separate experiment that used increasing number of Bif.BEVs. Created in BioRender.
Vishwakarma. V. (2025). Schematic illustration of gut commensal Bifidobacterium remotely enhance the therapeutic effect of anti-PD-1 in lung cancer. Created in BioRender.

What is claimed is:

1. A composition comprising:
an anti-cancer therapeutic agent; and
a bacteria-derived extracellular vesicle (BEV).

2. The composition of claim 1, wherein the anti-cancer therapeutic agent is one or more of: an alkylating agent, an antimetabolite, a plant alkaloid, an antitumor antibiotic, a topoisomerase inhibitor, a hormonal agent, a targeted therapeutic, an immunotherapy agent, a monoclonal antibody, a proteasome inhibitor, an epigenetic modifier, hydroxyurea, or L-asparaginase.

3. The composition of claim 1, wherein the BEV is derived from commensal bacteria.

4. The composition of claim 1, wherein the BEV is derived from one or more of: gut microbiota commensal bacteria, skin microbiota commensal bacteria, oral microbiota commensal bacteria, respiratory tract microbiota commensal bacteria, urogenital microbiota commensal bacteria, or nasal microbiota commensal bacteria.

5. The composition of claim 1, wherein the BEV is derived from one or more Bifidobacterium species.

6. The composition of claim 1, further comprising a pharmaceutically acceptable carrier, additive, excipient, or other component of a pharmaceutical composition.

7. The composition of claim 1, wherein the BEV includes lipoteichoic acid (LTA).

8. A method of treating a disease, comprising:
administering a bacteria-derived extracellular vesicle (BEV) to a subject having a disease in an amount sufficient to treat the disease.

9. The method of claim 8, wherein the disease is cancer.

10. The method of claim 8, wherein the BEV is administered with an anti-cancer therapeutic agent.

11. The method of claim 8, wherein the BEV is derived from commensal bacteria.

12. The method of claim 8, wherein the BEV is derived from one or more of: gut microbiota commensal bacteria, skin microbiota commensal bacteria, oral microbiota commensal bacteria, respiratory tract microbiota commensal bacteria, urogenital microbiota commensal bacteria, or nasal microbiota commensal bacteria.

13. The method of claim 8, wherein the BEV is derived from one or more Bifidobacterium species.

14. The method of claim 8, wherein the BEV is configured to regulate an immune checkpoint.

15. The method of claim 8, wherein the BEV is configured to inhibit upregulation or decrease regulation of programmed death-ligand 1 (PD-L1).

16. The method of claim 8, wherein the BEV is administered with an anti-programmed death-ligand 1 (anti-PD-L1) immune checkpoint inhibitor.

17. The method of claim 16, wherein the BEV and the anti-PD-L1 immune checkpoint inhibitor cause one or more of:
modulated cytokine levels, or
increased tumor infiltrating lymphocytes.

18. The method of claim 8, wherein the BEV is configured to cause one or more of: upregulation of major histocompatibility complex (MHC) peptide, increased interferon response, increased toll-like receptor 4 (TLR4) signaling, or increased inhibition of angiogenesis.

19. A method of increasing antitumor immune response in a subject with a disease, comprising:
administering an effective amount of bacteria-derived extracellular vesicle (BEV) and anti-programmed death-ligand 1 (anti-PD-L1) immune checkpoint inhibitor.

20. The method of claim 19, wherein the disease is cancer.

21. The method of claim 19, wherein the BEV is derived from commensal bacteria.

22. The method of claim 19, wherein the BEV is derived from one or more of: gut microbiota commensal bacteria, skin microbiota commensal bacteria, oral microbiota commensal bacteria, respiratory tract microbiota commensal bacteria, urogenital microbiota commensal bacteria, or nasal microbiota commensal bacteria.

23. The method of claim 19, wherein the BEV is derived from one or more Bifidobacterium species.

24. The method of claim 19, wherein administering the effective amount of the BEV and the anti-PD-L1 immune checkpoint inhibitor cause one or more of:
modulated cytokine levels, or
increased tumor infiltrating lymphocytes.

25. The method of claim 19, wherein the BEV is configured to cause one or more of: upregulation of major histocompatibility complex (MHC) peptide, increased interferon response, increased toll-like receptor 4 (TLR4) signaling, or increased inhibition of angiogenesis.

26. A kit for treating lung cancer, comprising:
commensal Bifidobacterium-derived extracellular vesicles (Bif.BEVs); and
one or more of an anti-programmed death 1 (anti-PD-1) immune checkpoint inhibitor or an anti-programmed death-ligand 1 (anti-PD-L1) immune checkpoint inhibitor.

27. The kit of claim 26, wherein the Bif.BEVs include lipoteichoic acid (LTA).

28. The kit of claim 26, wherein the Bif.BEVs are derived from gut microbiota commensal bacteria, skin microbiota commensal bacteria, oral microbiota commensal bacteria, respiratory tract microbiota commensal bacteria, urogenital microbiota commensal bacteria, or nasal microbiota commensal bacteria.

29. The kit of claim 26, wherein the anti-PD-1 immune checkpoint inhibitor includes one or more of nivolumab, pembrolizumab, cemiplimab, tislelizumab, dostarlimab, retifanlimab, and toripalimab.

30. The kit of claim 26, wherein the anti-PD-L1 immune check point inhibitor includes one or more of atezolizumab, avelumab, durvalumab, and cosibelimab.
